(19) United States
(12) Patent Application Publication
(10) Pub. No.: US 2006/0292203 A1
(43) Pub. Date: Dec. 28, 2006

(54) Methods and compositions for the treatment of ocular disorders

(75) Inventors: Luis A. Dellamary, San Diego, CA (US); Arek Tabak, San Diego, CA (US); Shiyin Yee, San Diego, CA (US)

Correspondence Address: DLA PIPER US LLP, 4365 EXECUTIVE DRIVE, SUITE 1100, SAN DIEGO, CA, 92121-2133, US

(73) Assignee: TargeGen, Inc., San Diego, CA (US)

(21) Appl. No.: 11/449,219

(22) Filed: Jun. 7, 2006

Related U.S. Application Data
(60) Provisional application No. 60/689,111, filed on Jun. 8, 2005.
(60) Provisional application No. 60/763,537, filed on Jan. 30, 2006.

(51) Int. Cl. A61K 31/506; A61F 2/00

(52) U.S. Cl. 424/427; 514/275

(57) ABSTRACT

The invention provides methods and compositions for the delivery of lipophilic drugs that are useful for the treatment of various ophthalmological diseases, disorders, and pathologies, including the treatment of age-related macular degeneration, diabetic retinopathy, diabetic macular edema, cancer, and glaucoma.

### RELATED APPLICATION DATA

This application claims priority under 35 U.S.C. § 119(e) to U.S. Patent Application Ser. Nos. 60/689,111, filed Jun. 8, 2005 and 60/763,537 filed Jan. 30, 2006, the entire content of each of which is herein incorporated by reference in its entirety.

### BACKGROUND

1. Field of the Invention

The present invention relates generally to ophthalmic conditions and more specifically to the use of compositions formulated for ophthalmic delivery, especially formulations for delivery to the back of the eye.

2. Background of the Invention

One of the difficulties that often arises in treating ocular diseases is the inefficiency of delivering therapeutic agents intraocularly. When a drug is delivered intraocularly, it typically clears rapidly from the ocular tissues. Because of this inherent difficulty of delivering drugs into the eye, successful treatment of ocular diseases can often be difficult.

Due to the anatomical structure of the eye and its physiological nature, targeting a drug to the appropriate site of action is usually one of the greatest challenges in drug delivery to the eye.

Traditionally, topical ophthalmic solutions, suspensions and semisolids have been used for the ocular therapeutic preparations. A disadvantage associated with using such conventional dosage forms is that they often exhibit insufficient ocular bioavailability. More recently, other ocular drug delivery systems have been developed. Some of these systems include controlled release systems such as ocular inserts, nanoparticles, mucoadhesive polymers, water soluble drug-loaded films and liposomal dosage forms. The latter type has shown some promise, but exhibited inadequate stability of the encapsulated drug. In addition, even though liposomal formulations have been shown to be effective in delivering drug to the eye via topical instillation, they have not been able to describe the parameter necessary to be able to efficiently deliver drug to the back of the eye with a drug delivery system suitable for commercial use. Accordingly there has been only limited use of liposomal dosage forms.

Many currently available ophthalmic drugs have a fair to high water solubility, while the drugs with very limited solubility or those considered insoluble in water have been often considered unusable and, in some cases, discarded as to further development. Some of these lipophilic and water insoluble drugs can posses desirable therapeutic properties, but, due to their solubility properties, they can be rendered useless. Drugs in this class can have a high affinity for target cell membranes and lipophilic tissues, but are difficult to deliver due to their low water solubility and difficulties arising during attempts to administer them. Some of these lipophilic and water insoluble drugs can have a high affinity for phospholipids rendering them suitable to be delivered via liposomes or phospholipid compositions where the drug is not encapsulated in the aqueous core of the liposome but rather forms an integral part of the phospholipid matrix or the phospholipid membrane.

While the general process of absorption in the eye may not be completely elucidated, there are well known relationships between molecular properties, transport and penetration, which play a role in the process of absorption. It is known that there is a relationship between the permeability of drugs across biological membranes and the octanol-water partition coefficient. A LogP of 2.9 was shown to be optimal for beta-blocking agents and their corneal permeabilities using excised rabbit corneas (see, Schoenwald, et al., 1983, J. Pharm. Sci., 72:1266). Unfortunately the delivery of such lipophilic drugs is limited due to its low water solubility or inappropriate drug dosage form, in particular when delivered to the eye.

Accordingly, it is desirable to be able to prepare a formulation of drugs that both have affinity to phospholipids and are water insoluble, into lipid vesicles of lipid compositions composed of at least one phospholipid. Such compositions have not been previously elaborated but are needed because they possess high efficiency of loading and negligible “leakage” due to high partitioning of the drug into the lipid compared to the water.

### SUMMARY

According to one embodiment of the present invention, compositions for treatment of various ocular diseases are provided, the compositions comprising a drug or its prodrug, and a pharmaceutically acceptable carrier for ophthalmic delivery, wherein the drug is not a steroidal molecule. The drug or its prodrug has a polar surface area not exceeding about 150 Å², such as less than about 120 Å², for example, not exceeding about 100 Å². The drug or its prodrug can further have a water solubility of less than about 0.1 mg/mL at a pH range of 4-8, such as less than about 0.05 mg/mL at a pH range of 4-8, for example, less than about 0.01 mg/mL at a pH range of 4-8. The drug or its prodrug can additionally have a cLogD of at least about 0.5 at pH of 7.4, such as at least about 1, for example, at least 2. The drug or its prodrug can further have a molecular weight not exceeding about 1,000 Daltons, such as not exceeding about 900 Daltons, for example, not exceeding about 800 Daltons. Physical and chemical properties of some selected limiting, drugs and prodrugs of the invention or known in the art, are illustrated in Table 1.

TABLE 1
Physicochemical properties of some selected drugs or prodrugs

| | | | | | | mg/mL | Phospholipid | |
|---|---|---|---|---|---|---|---|---|
| Compound and Properties | | | | | | Water | affinity | |
| | | Molar | Molar | | pH = 7.4 | solubility | PC: Drug | Polariz- |
| | PSA | Refractivity | volume | IR | cLogD | (pH 5) | (molar) | ability |
| Acetazolamide | 151.66 | 45.95 | 127.39 | 1.64 | −0.55 | <1 | | 18.22 |
| Brimonidine | 62.2 | 68.42 | 160.3 | 1.8 | −0.39 | >2 | | 27.12 |
| III | 119.48 | 134.24 | 348.06 | 1.7 | −0.37 | | | 53.22 |
| Su11248¹⁾ | 77.23 | 112.52 | 324.06 | 1.61 | 0.85 | >1 | 30:1 | 44.61 |
| XXI*⁾ | 144.06 | 97.88 | 228.43 | 1.8 | 0.93 | <<0.1 | >100:1 | 38.8 |
| propanolol | 41.49 | 78.99 | 237.16 | 1.58 | 1.37 | ˜0.1 | | 31.31 |
| XII | 139.74 | 145.38 | 385.74 | 1.68 | 0.14 | <<0.1 | 100:1 | 57.63 |
| Tropicamide | 53.43 | 82.2 | 244.83 | 1.59 | 1.15 | <1 | >2:1 | 32.59 |
| I | 79.38 | 127.51 | 341 | 1.67 | 1.21 | <<0.1 | | 50.55 |
| AP23464²⁾ | 102.74 | 133.99 | 351.01 | 1.69 | 1.42 | >0.1 | | 53.12 |
| CGP76775³⁾ | 89.43 | 127.62 | 338.86 | 1.68 | 1.92 | <0.1 | | 50.59 |
| XVII | 89.47 | 144.73 | 396.71 | 1.65 | 2.31 | <<0.1 | 30:1 | 57.38 |
| TAA⁴⁾ | 93.06 | 109.41 | 324.83 | 1.59 | 2.5 | <0.1 | | 43.37 |
| XV | 128.72 | 146.74 | 388.34 | 1.68 | 2.61 | <<0.1 | | 58.17 |
| VII | 128.72 | 143.84 | 384.1 | 1.67 | 2.63 | <<0.1 | 30:1 | 57.02 |
| V | 83.4 | 135.27 | 357.51 | 1.68 | 2.71 | <<0.1 | 30:1 | 53.63 |
| V-propionate | 89.47 | 149.37 | 413.22 | 1.64 | 2.84 | <<0.1 | | 59.21 |
| XVI | 128.72 | 148.67 | 396.05 | 1.67 | 2.94 | <<0.1 | | 58.94 |
| PP1⁵⁾ | 69.62 | 83.46 | 228.25 | 1.65 | 3.11 | <0.1 | | 33.09 |
| XX | 89.47 | 153.96 | 430.1 | 1.63 | 3.19 | <<0.1 | 10:1 | 61.03 |
| VIII | 102.36 | 163.13 | 435.72 | 1.67 | 3.24 | <<0.1 | | 64.67 |
| AZM475271⁶⁾ | 77.97 | 127.97 | 353.72 | 1.64 | 3.26 | <<0.1 | 50.73 | |
| XIII | 108.49 | 146.73 | 406.27 | 1.64 | 3.48 | <<0.1 | <20:1 | 58.17 |
| X | 89.47 | 158.6 | 446.28 | 1.63 | 3.54 | <<0.1 | 10:1 | 62.87 |
| IV | 63.17 | 138.15 | 379.68 | 1.65 | 3.56 | <<0.1 | 10:1 | 54.77 |
| XI | 63.17 | 138.29 | 371.03 | 1.67 | 3.61 | <<0.1 | 10:1 | 54.82 |
| Vatalanib | 50.7 | 101.95 | 260.61 | 1.71 | 3.79 | >2 | Not stable | 40.42 |
| XVIII | 134.79 | 173.51 | 469.09 | 1.66 | 4.43 | <<0.1 | 30:1 | 68.78 |
| VI | 89.47 | 165.04 | 442.5 | 1.67 | 4.5 | <<0.1 | 10:1 | 65.43 |
| Dexamethasone | 100.9 | 123.71 | 382.35 | 1.56 | 4.55 | <<0.1 | | 49.04 |
| valerate | | | | | | | | |
| SKI606⁷⁾ | 82.88 | 141.92 | 388.35 | 1.65 | 4.63 | <1 | 10:1 | 56.26 |
| XIX | 89.47 | 169.86 | 458.77 | 1.66 | 4.96 | <<0.1 | | 67.34 |
| PD180970⁸⁾ | 58.12 | 111.04 | 296.16 | 1.67 | 5.13 | <<0.1 | | 44.02 |
| Cholesterol | 20.23 | 119.97 | 391.43 | 1.53 | 9.85 | <<1 | >2:1 | 47.56 |
| Tacrolimus | 178.36 | 214.13 | 673.12 | 1.55 | 3.96 | <0.1 | | 84.89 |
| (FK506) | | | | | | | | |
| cyclosporine A | 278.8 | 328.83 | 1183.63 | 1.47 | 3.35 | >1 | | 130.36 |

Notes.
Roman numerals refer to the compounds shown in the application under those numerals
*⁾XXI reers to the compound XXI:

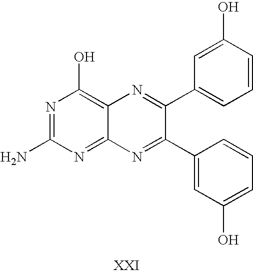

¹⁾Sugen11248 refers to the compound available from
²⁾AP23464 refers to the compound available from
³⁾CGP76775 refers to the compound available from
⁴⁾TAA refers to the compound available from
⁵⁾PP1 refers to the compound available from
⁶⁾AZM475271 refers to the compound available from
⁷⁾SKI606 refers to the compound available from Smith Kline Co.
⁸⁾PD180970 refers to the compound available from

According to another embodiment of the present invention, the compositions include an active compound or drug having the structure A:

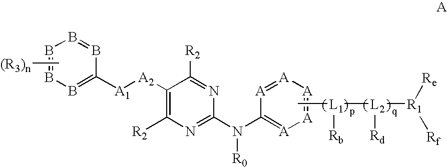

In structure A, each of A can be, independently, one of CH, N, NH, O, S, or a part of a ring fusion to form a second ring, wherein the second ring can be an aromatic, a heteroaromatic, a bicyclic aromatic, or a bicyclic aromatic heterocyclic ring;

each of B can be, independently CH, or a part of a ring fusion to form a second ring, wherein the second ring can be an aromatic, a bicyclic aromatic, or a bicyclic with only the first ring being aromatic;

A₁ can be one of NRₐ, C(O), S(O), S(O)₂, P(O)₂, O, S, or CRₐ, where R can be one of H, lower alkyl, branched alkyl, hydroxyalkyl, aminoalkyl, thioalkyl, alkylhydroxyl, alklythiol, or alkylamino, and wherein a=1, if A₁ is NRₐ, and a=2, if A₁ is CRₐ;

A₂ can be one of NR, C(O), S(O), S(O)₂, P(O)₂, O, or S, with the proviso that the connectivity between A₁ and A₂ is chemically correct;

R₀ can be one of H, lower alkyl, or branched alkyl;

L₁ can be one of a bond, O, S, C(O), S(O), S(O)₂, NRₐ, C₁-C₆ alkyl; L₂ can be one of a bond, O, S, C(O), S(O), S(O)₂, C₁-C₆, NRₐ; or L₁ and L₂ taken together can be a bond;

each of R_{b}, R_{d}, Rₑ, R_{f} either is absent or is independently one of H, C₁-C₆ alkyl, cycloalkyl, branched alkyl, hydroxy alkyl, aminoalkyl, thioalkyl, alkylhydroxyl, alkklythiol, or alkylamino;

each of p, q, m, r is independently an integer having value from 0 to 6;

R_{b} and R_{d} taken together can be one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NRₐ—(CH₂)ₘ, or (CH₂)ᵣ—O—(CH₂)ₘ; or

R_{b} and Rₑ taken together can be one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NRₐ—(CH₂)ₘ, or (CH₂)ᵣ—O—(CH₂)ₘ;

or R_{d} and R_{f} taken together can be one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NRₐ—(CH₂)ₘ, or (CH₂)ᵣ—O—(CH₂)ₘ; or

R_{b} and R_{f} taken together can be one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NRₐ—(CH₂)ₘ, or (CH₂)ᵣ—O—(CH₂)ₘ; or

R_{d} and Rₑ taken together can be one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NRₐ—(CH₂)ₘ, and (CH₂)ᵣ—O—(CH₂)ₘ;

R₁ can be one of (CRₐ)ₘ, O, N, S, C(O)(O)R′, C(O)N(R′)₂, SO₃R′, OSO₂R′, SO₂R′, SOR′, PO₄R′, OPO₂R′, PO₃R′, PO₂R′, or a 3-6 membered heterocycle with one or more heterocyclic atoms, wherein R′ can be one of hydrogen, lower alkyl, alkyl-hydroxyl, or can form a closed 3-6 membered heterocycle with one or more heterocyclic atoms, branched alkyl, branched alkyl hydroxyl, where each R′ is independent in case there is more than one R′;

R₂ can be one of hydrogen, alkyl, branched alkyl, phenyl, substituted phenyl, halogen, alkylamino, alkyloxo, CF₃, sulfonamido, substituted sulfonamido, alkyoxy, thioalkyl, sulfonate, sulfonate ester, phosphate, phosphate ester, phosphonate, phosphonate ester, carboxo, amido, ureido, substituted carboxo, substituted amido, substituted ureido, or 3-6 membered heterocycle with one or more hetrocyclic atoms, with the further proviso that either one or two substituents R₂ can be present in the ring, and if more than one substituent R₂ are present, each of the substituents can be the same or different;

R₃ can be one of hydrogen, alkyl, branched alkyl, alkoxy, halogen, CF₃, cyano, substituted alkyl, hydroxyl, alklylhydroxyl, thiol, alkylthiol, thioalkyl, amino, or aminoalkyl; and

n is an integer that can have value between 1 and 5, with the further proviso that if n≧2, then each group R₃ is independent of the other groups R₃.

According to yet another embodiment of the present invention, the composition includes an active compounds or drug having the structure B:

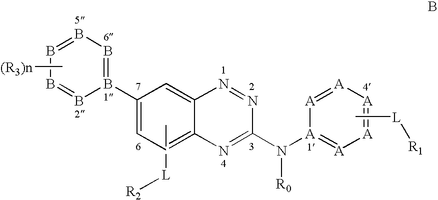

In the structure B, each of A can be independently selected from a group consisting of (CH)₀₋₁, N, NH, O, S, and a part of a ring fusion to form a second ring, where the second ring is an aromatic, a heteroaromatic, a bicyclic aromatic, a bicyclic aromatic heterocyclic ring, or a bicyclic with only the first ring being aromatic or heteroaromatic;

each of B can be independently selected from a group consisting of (CH)₀₋₁, N, NH, O, S, and a part of a ring fusion to form a second ring, where the second ring is an aromatic, a heteroaromatic, a bicyclic aromatic, a bicyclic aromatic heterocyclic ring, or a bicyclic with only the first ring being aromatic or heteroaromatic, with the further proviso that if each B is (CH)₀, R₃ is bonded directly to the adjacent ring.

R₀ can be selected from a group consisting of H and lower alkyl;

L can be selected from a group consisting of a bond, and a substituted or unsubstituted alkyl, alkenyl, or alkynyl linking moiety;

R₁ can be selected from a group consisting of C(R′)₃, OR′, N(R′)₂, NR′C(O)R′, NR′C(O)O(R′), NR′C(O)N(R′)₂, SR′, C(O)(O)R′, C(O)R′, C(O)N(R′)₂, SO₃R′, OSO₂R′, SO₂R′, SOR′, S(O)N(R′)₂, OS(O)(O)N(R′)₂, S(O)(O)N(R′)₂, S(O)N(R′)₂, PO₄R′, OPO₂R′, PO₃R′, PO₂R′, and a 3-6 membered heterocycle with one or more heterocyclic atoms with each heteroatom independently being capable of carrying any R′ group on it, wherein R′ is selected from a group consisting of hydrogen, lower an alkyl, a substituted alkyl, an alkyl-hydroxyl, a substituted alkyl-hydroxyl, a thiol-alkyl, a thiol-substituted alkyl, an alkyl-thiol, a substituted alkyl-thiol, an aminoalkyl, an amino-substituted alkyl, an alkylamino, a substituted alkyl-amino, a branched alkyl, a branched substituted alkyl, a branched alkyl hydroxyl, a branched substituted alkyl hydroxyl, a branched thio-alkyl, a branched thio-substituted alkyl, a branched alkyl-thiol, a branched substituted alkyl-thiol, a branched aminoalkyl, a branched amino-substituted alkyl, a branched alkylamino, a branched substituted alkyl-amino, and a closed 3-6 membered carbocycle or heterocycle, wherein a substitutent in any of said substituted alkyls includes said closed 3-6 membered carbocycle or heterocycle, with the further proviso that each heteroatom in the 3-6 membered heterocycle being capable of carrying any R′ group on it, with the further proviso that the substitution in any of said substituted alkyls includes any R′ group connected to said alkyls via an atom other than carbon or via carbon, and wherein each R′ is independent in case there is more than one R′;

R₂ is a substitutent situated at position 5, 6 or 8 of the ring, wherein R₂ can be selected from a group consisting of methyl, ethyl, n-propyl, iso-propyl, n-butyl, iso-butyl, tert-butyl, iso-pentyl, phenyl, substituted phenyl, halogen, branched or unbranched alkylamino, branched or unbranched aminoalkyl, branched or unbranched alkyloxo, branched or unbranched oxyalkyl, branched or unbranched thioalkyl, branched or unbranched alkylthiol, CF₃, sulfonamido, substituted sulfonamido, sulfonate, sulfonate ester, phosphate, phosphate ester, phosphonate, phosphonate ester, carboxo, amido, ureido, substituted carboxo, substituted amido, substituted ureido, or a 3-6 membered carbocycle or heterocycle attached to positions 5, 6 or 8 directly or through group L, each heteroatom independently being capable of carrying any group R₂, with the further proviso that either one, two or three substituents R₂ are present in the ring, each of the substituents R₂ being the same or different;

R₃ can be selected from a group consisting of hydrogen, alkyl, alkoxy, halogen, CF₃, cyano, substituted alkyl, or hydroxyl, aryl, substituted aryl, heteroaryl, substituted heteroaryl, heterocycle, C(R″)₃, OR″, N(R″)₂, NR″C(O)R″, NR″C(O)NR″, R″, C(O)(O)R″, OC(O)R″, C(O)N(R″)₂, C(O), C(O)R″, OC(O)N(R″)₂, SO₃R″, OSO₂R″, SO₂R″, SOR″, PO₄R″, OPO₂R″, PO₃R″, PO₂R″, wherein R″ is hydrogen, aryl, substituted aryl, heteroaryl, substituted heteroaryl, lower alkyl, branched lower alkyl, alkyl-hydroxyl, branched alkyl-hydroxyl, amino-alkyl, branched amino-alkyl, alkyl-amino, branched alkyl-amino, thiol-alkyl, branched thiol-alkyl, alkyl-thiol, branched thiol-alkyl, or may form a closed 3-6 membered heterocycle with one or more heterocyclic atoms, branched alkyl, branched alkyl hydroxyl, where each R″ is independent in case there is more than one R″;

n is an integer having the value between 1 and 5, with the further proviso that if n≧2, then each group R₃ is independent of the other groups R₃,

with the further proviso that if each A is (CH)₀, L is a bond,

with the further proviso that if each B is (CH)₀, R₃ can be any substitutent described above, other than hydrogen, bonded directly to the position 7 of the adjacent ring; and pharmaceutically acceptable salts, hydrates, solvates, crystal forms, N-oxides, and individuals diastereomners thereof.

According to another embodiment of the present invention, a method for treating an ophthalmological condition in a subject is provided, the method including administering to a subject in need thereof a therapeutically effective amount of a composition including an active compound or drug having a) a polar surface area not exceeding about 150 Å²; b) a water solubility of less than about 0.1 mg/mL at a pH range of 4-8; c) a cLogD of at least about 0.5 at pH of 7.4; and d) a molecular weight not exceeding about 1,000 Daltons, with the proviso that the drug is not a steroidal molecule, including compounds exemplified by the structure set forth in A or B herein, thereby treating the condition.

According to yet another embodiment of the present invention, a method for preparing a composition is provided, the composition including an active compound or drug having the structure A or B. The method includes dissolving or partially dissolving the compound or drug in the presence or absence of an organic solvent; mixing with an aqueous colloidal suspension containing the polymer base carrier; removing the solvent; adding osmotic agents; and adjusting pH to a value making the composition suitable for administration.

According to another embodiment of the present invention, a method of delivering a compound to the back of an eye is provided, the method including preparing a formulation including a therapeutically effective amount of an active compound or drug having the structure A or B, and delivering the formulation to an eye of a subject in need of such delivery.

According to another embodiment of the present invention, a method of identifying a compound suitable for delivery to the eye is provided, the method including administering a compound by eye drop administration and observing the distribution of the compound in the eye following eye drop administration, wherein the compound is not a steroidal molecule, thereby identifying a compound suitable for delivery to the eye. A compound used in such a method typically has a polar surface area not exceeding about 150 Å², such as less than about 120 Å², for example, not exceeding about 100 Å². The compound further has a water solubility of less than about 0.1 mg/mL at a pH range of 4-8, such as less than about 0.05 mg/mL at a pH range of 4-8, for example, less than about 0.01 mg/mL at a pH range of 4-8. The compound additionally has a cLogD of at least about 0.5 at pH of 7.4, such as at least about 1, for example, at least 2. The compound further has a molecular weight not exceeding about 1,000 Daltons, such as not exceeding about 900 Daltons, for example, not exceeding about 800 Daltons.

According to yet another embodiment of the present invention, an article of manufacture is provided, the article of manufacture including a vial containing a composition including a therapeutically effective amount of an active compound or drug having the structure A or B, and further including instructions for administration of the composition.

### BRIEF DESCRIPTION OF THE DRAWINGS

FIG. 1 is a graph showing eyedrop administration of invention compounds blocks VEGF induced permeability in the eye.

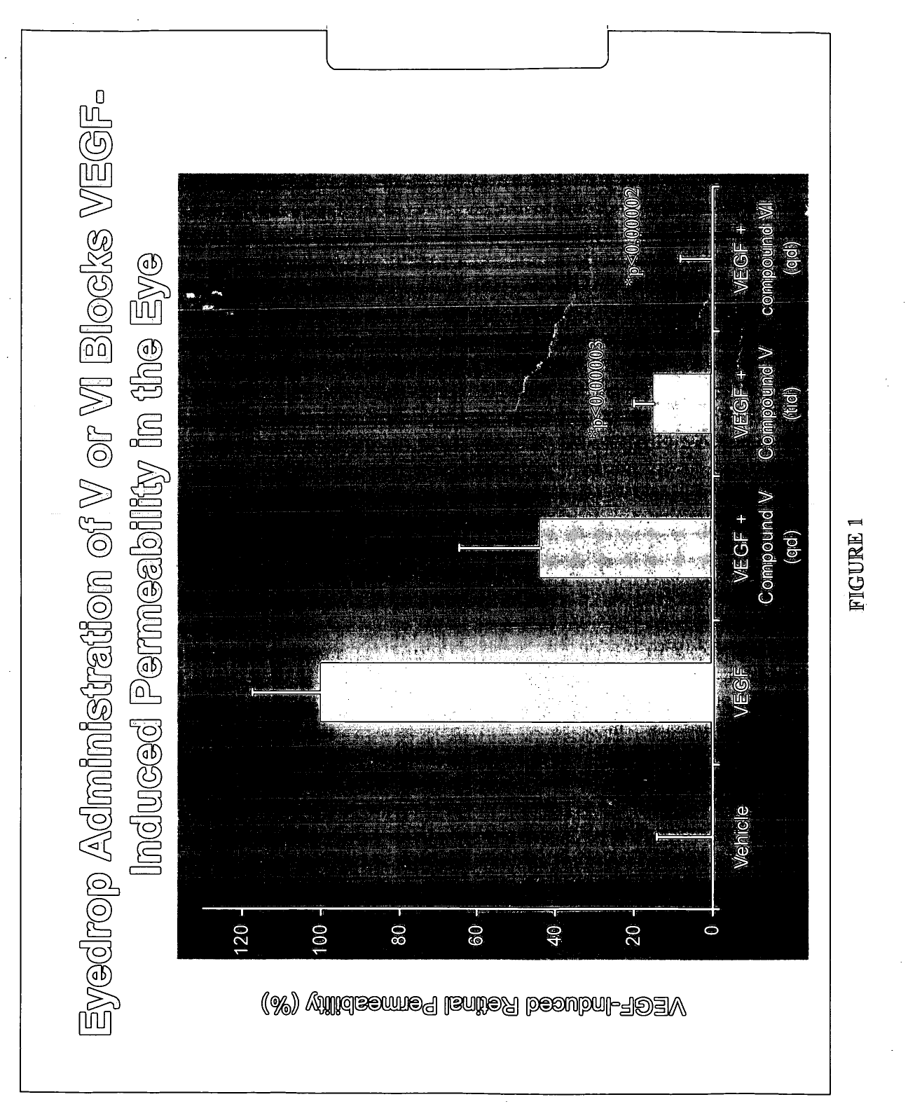

FIG. 2 is a graph showing topical administration of compound VI prevents choroidal neovascularization (CNV) in the eye in a laser-induced CNV model.

FIG. 3 is pharmacokinetics (PK) data with a graph showing back of the eye exposure of compound VI instilled topically (eye drop) in C57BL/6 mice.

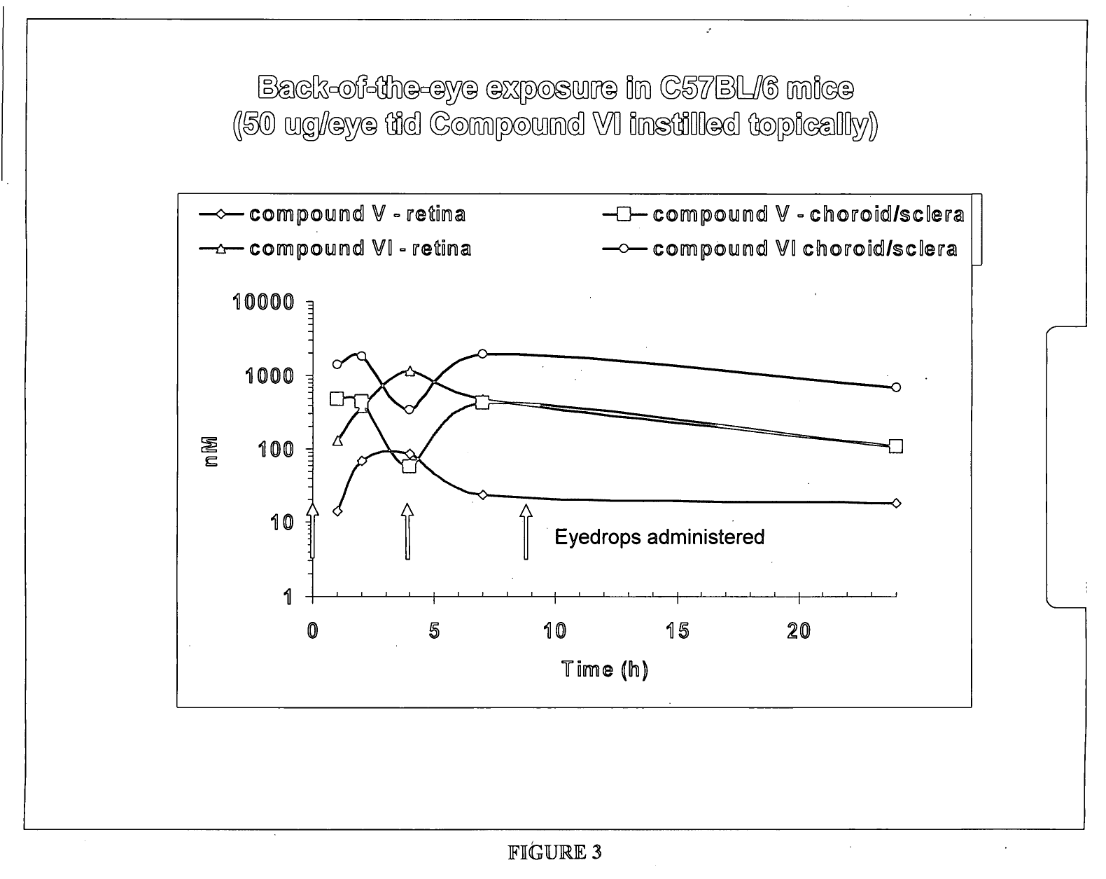

FIG. 4 is a PK data graph and table showing concentrations of compound V or VI in the tissues at the back of the eye following topical instillation (eye drop) of compound V or VI in mice.

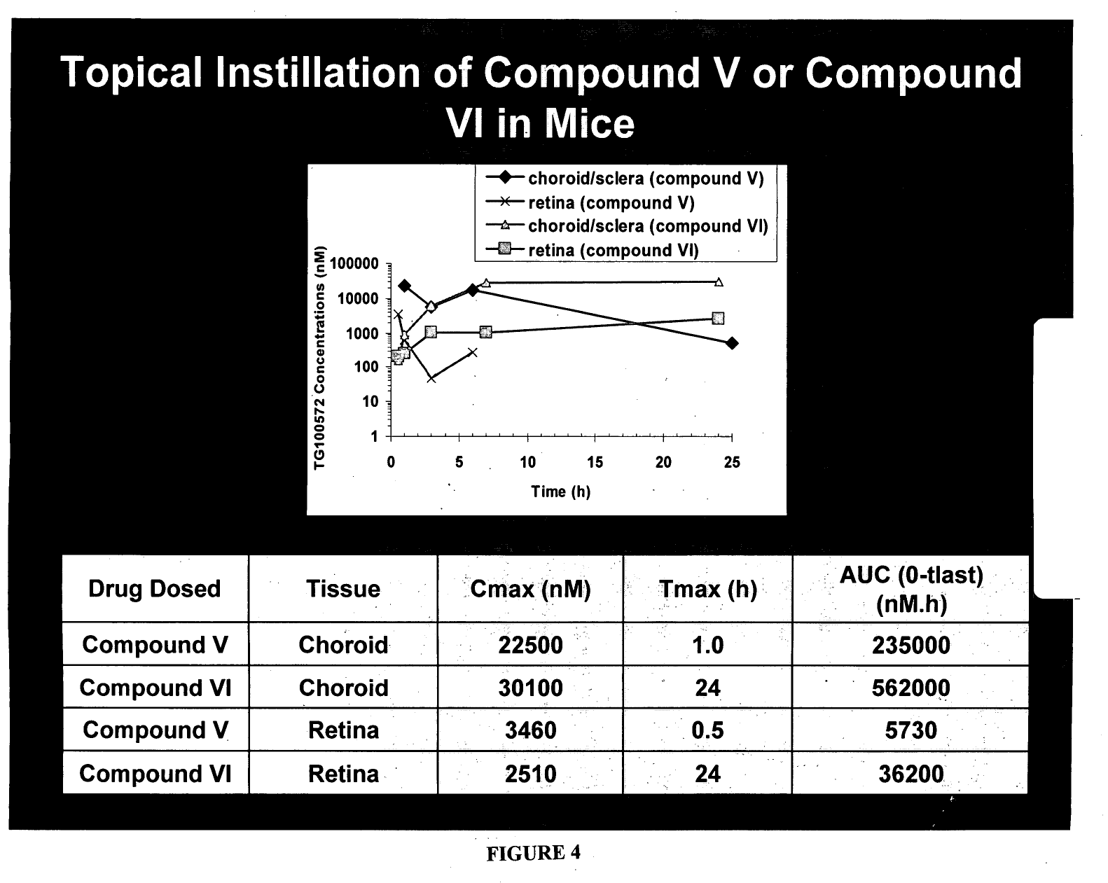

FIG. 5 is a PK data graph and table showing steady-state choroidal concentrations of compound V following topical instillation of compound VI in three different species—rabbit, dog and minipig.

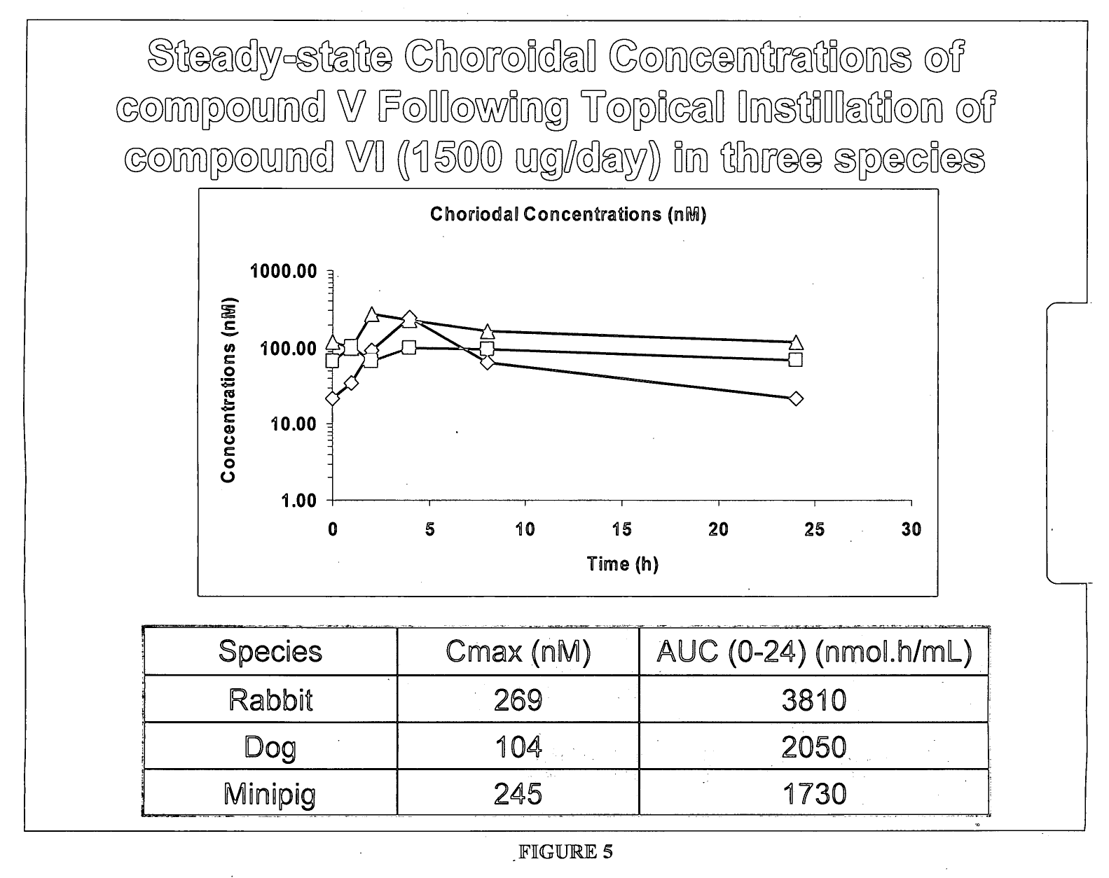

FIG. 6 is a PK data table showing ocular exposure in the back of the eye following topical instillation of compound VI in Dutch-Belted rabbits.

### DETAILED DESCRIPTION

The following terminology and definitions apply as used in the present application, generally in conformity with the terminology recommended by the International Union of Pure and Applied Chemistry (IUPAC):

The term “heteroatom” refers to any atom other than carbon, for example, N, O, or S.

The term “aromatic” refers to a cyclically conjugated molecular entity with a stability, due to delocalization, significantly greater than that of a hypothetical localized structure, such as the Kekule structure.

The term “heterocyclic,” when used to describe an aromatic ring, refers to the aromatic rings containing at least one heteroatom, as defined above.

The term “heterocyclic,” when not used to describe an aromatic ring, refers to cyclic (i.e., ring-containing) groups other than aromatic groups, the cyclic group being formed by between 3 and about 14 carbon atoms and at least one heteroatom described above.

The term “substituted heterocyclic” refers, for both aromatic and non-aromatic structures, to heterocyclic groups further bearing one or more substituents described below.

The term “alkyl” refers to a monovalent straight or branched chain hydrocarbon group having from one to about 12 carbon atoms, for example, methyl, ethyl, n-propyl, iso-propyl, n-butyl, iso-butyl, tert-butyl, n-pentyl (also known as n-amyl), n-hexyl, and the like. The term “lower alkyl” refers to alkyl groups having from 1 to about 6 carbon atoms.

The term “substituted alkyl” refers to alkyl groups further bearing one or more substituents such as hydroxy, alkoxy, mercapto, cycloalkyl, substituted cycloalkyl, heterocyclic, substituted heterocyclic, aryl, substituted aryl, heteroaryl, substituted heteroaryl, aryloxy, substituted aryloxy, halogen, cyano, nitro, amino, amido, aldehyde, acyl, oxyacyl, carboxyl, sulfonyl, sulfonamide, sulfuryl, and the like.

The term “alkenyl” refers to straight-chained or branched hydrocarbyl groups having at least one carbon-carbon double bond, and having between about 2 and about 12 carbon atoms, and the term “substituted alkenyl” refers to alkenyl groups further bearing one or more substituents described above.

The term “alkynyl” refers to straight-chained or branched hydrocarbyl groups having at least one carbon-carbon triple bond, and having between about 2 and about 12 carbon atoms, and the term “substituted alkynyl” refers to alkynyl groups further bearing one or more substituents described above.

The term “aryl” refers to aromatic groups having between about 5 and about 14 carbon atoms and the term “substituted aryl” refers to aryl groups further bearing one or more substituents described above.

The term “heteroaryl” refers to aromatic rings, where the ring structure is formed by between 3 and about 14 carbon atoms and by at least one heteroatom described above, and the term “substituted heteroaryl” refers to heteroaryl groups further bearing one or more substituents described above.

The term “alkoxy” refers to the moiety —O-alkyl, wherein alkyl is as defined above, and the term “substituted alkoxy” refers to alkoxy groups further bearing one or more substituents described above.

The term “cycloalkyl” refers to alkyl groups having between 3 and about 8 carbon atoms arranged as a ring, and the term “substituted cycloalkyl” refers to cycloalkyl groups further bearing one or more substituents described above.

The term “alkylaryl” refers to alkyl-substituted aryl groups and the term “substituted alkylaryl” refers to alkylaryl groups further bearing one or more substituents described above.

The term “arylalkyl” refers to aryl-substituted alkyl groups and the term “substituted arylalkyl” refers to arylalkyl groups further bearing one or more substituents described above.

The term “arylalkenyl” refers to aryl-substituted alkenyl groups and the term “substituted arylalkenyl” refers to arylalkenyl groups further bearing one or more substituents described above.

The term “arylalkynyl” refers to aryl-substituted alkynyl groups and the term “substituted arylalkynyl” refers to arylalkynyl groups further bearing one or more substituents described above.

The term “arylene” refers to divalent aromatic groups having between 5 and about 14 carbon atoms and the term “substituted arylene” refers to arylene groups further bearing one or more substituents described above.

The term “kinase” refers to any enzyme that catalyzes the addition of phosphate groups to a protein residue; for example, serine and threonine kinases catalyze the addition of phosphate groups to serine and threonine residues.

The term “therapeutically effective amount” refers to the amount of the compound or pharmaceutical composition that will elicit the biological or medical response of a tissue, system, animal or human that is being sought by the researcher, veterinarian, medical doctor or other clinician, e.g., restoration or maintenance of vasculostasis or prevention of the compromise or loss or vasculostasis; reduction of tumor burden; reduction of morbidity and/or mortality.

The term “pharmaceutically acceptable” refers to the fact that the carrier, diluent or excipient must be compatible with the other ingredients of the formulation and not deleterious to the recipient thereof.

The terms “administration of a compound” or “administering a compound” refer to the act of providing a compound of the invention or pharmaceutical composition to the subject in need of treatment.

The term “antibody” refers to intact molecules of polyclonal or monoclonal antibodies, as well as fragments thereof, such as Fab and F(ab′)₂, Fv and SCA fragments which are capable of binding an epitopic determinant.

The term “vasculostasis” refers to the maintenance of the homeostatic vascular functioning leading to the normal physiologic functioning.

The term “vasculostatic agents” refers to agents that seek to address conditions in which vasculostasis is compromised by preventing the loss of or restoring or maintaining vasculostasis.

The term “clogD” refers to the terminology that is used in any of the following software packages of the following companies: (1) ACD labs (Toronto Canada) ACD/physchem batch package or similar; or 2) Comgenex/Compudrug (Sedona Ariz.) Pallas software or similar; or (3) Syracuse Research Corporation (Syracuse N.Y.) KOWWIN software or similar.

Embodiments of the present invention describe pharmaceutical compositions including drugs (active compounds) effective for treating ocular disorders and pharmaceutically acceptable carriers. The active compounds included in the compositions can be distributed to, and are effective for treating of, ocular disorders, including ocular disorders the treatment of which requires drugs or prodrugs to reach the back of the eye. The drug that can be used is not a steroidal molecule. Among other requirements to the drugs that can be included in the compositions of the current invention are the following:

(a) the drug or its prodrug can have a polar surface area not exceeding about 150 Å², such as less than about 120 Å², for example, not exceeding about 100 Å²;

(b) the drug or its prodrug can further have a water solubility of less than about 0.1 mg/mL at a pH range of 4-8, such as less than about 0.05 mg/mL at a pH range of 4-8, for example, less than about 0.01 mg/mL at a pH range of 4-8;

(c) the drug or its prodrug can additionally have a cLogD of at least about 0.5 at pH of 7.4, such as at least about 1, for example, at least 2;

(d) the drug or its prodrug can further have a molecular weight not exceeding about 1,000 Daltons, such as not exceeding about 900 Daltons, for example, not exceeding about 800 Daltons.

The drugs suitable for the applications according to the present invention can be are any of antiallergics, antimigraine, antianemics, bronchodilators, analgesics, antibiotics, leukotriene inhibitors or antagonists, antihistamines, non-steroidal anti-inflammatories, antineoplastics, anticholinergics, anesthetics, anti-tuberculars, cardiovascular agents, lectins, peptides, and combinations thereof.

Illustrative compounds that satisfy the above-described requirements are disclosed below. According to an embodiment of the invention, pyrimidine-derived compounds having the structure A, or pharmaceutically acceptable salts, hydrates, solvates, crystal forms, N-oxides, and individuals diastereomers thereof, are provided for treatment of various ocular diseases, disorders, and pathologies.

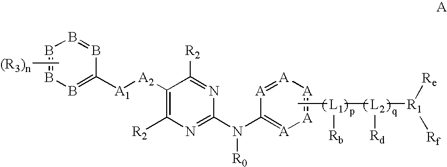

In the structure A, each of A can be, independently, one of CH, N, NH, O, S, or a part of a ring fusion to form a second ring, wherein the second ring can be an aromatic, a heteroaromatic, a bicyclic aromatic, or a bicyclic aromatic heterocyclic ring;

each of B can be, independently CH, or a part of a ring fusion to form a second ring, wherein the second ring can be an aromatic, a bicyclic aromatic, or a bicyclic with only the first ring being aromatic;

A₁ can be one of NRₐ, C(O), S(O), S(O)₂, P(O)₂, O, S, or CRₐ, where R can be one of H, lower alkyl, branched alkyl, hydroxyalkyl, aminoalkyl, thioalkyl, alkylhydroxyl, alklythiol, or alkylamino, and wherein a=1, if A₁ is NRₐ, and a=2, if A₁ is CRₐ;

A₂ can be one of NR, C(O), S(O), S(O)₂, P(O)₂, O, or S, with the proviso that the connectivity between A₁ and A₂ is chemically correct;

R₀ can be one of H, lower alkyl, or branched alkyl;

L₁ can be one of a bond, O, S, C(O), S(O), S(O)₂, NRₐ, C₁-C₆ alkyl; L₂ can be one of a bond, O, S, C(O), S(O), S(O)₂, C₁-C₆, NRₐ; or L₁ and L₂ taken together can be a bond;

each of R_{b}, R_{d}, Rₑ, R_{f} either is absent or is independently one of H, C₁-C₆ alkyl, cycloalkyl, branched alkyl, hydroxy alkyl, aminoalkyl, thioalkyl, alkylhydroxyl, alkklythiol, or alkylamino;

each of p, q, m, r is independently an integer having value from 0 to 6;

R_{b} and R_{d} taken together can be one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NRₐ—(CH₂)ₘ, or (CH₂)ᵣ—O—(CH₂)ₘ; or

R_{b} and Rₑ taken together can be one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NRₐ—(CH₂)ₘ, or (CH₂)ᵣ—O—(CH₂)ₘ;

or R_{d} and R_{f} taken together can be one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NRₐ—(CH₂)ₘ, or (CH₂)ᵣ—O—(CH₂)ₘ; or

R_{b} and R_{f} taken together can be one of (CH₂)ₘ, (CH₂)ᵣ—S—(CH₂)ₘ, (CH₂)ᵣ—SO(CH₂)ₘ, (CH₂)ᵣ—SO₂—(CH₂)ₘ, (CH₂)ᵣ—NRₐ—(CH₂)ₘ, or (CH₂)ᵣ—O—(CH₂)ₘ; or

R_{d} and Rₑ taken together can be one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NRₐ—(CH₂)ₘ, and (CH₂)ᵣ—O—(CH₂)ₘ;

R₁ can be one of (CRₐ)ₘ, O, N, S, C(O)(O)R′, C(O)N(R′)₂, SO₃R′, OSO₂R′, SO₂R′, SOR′, PO₄R′, OPO₂R′, PO₃R′, PO₂R′, or a 3-6 membered heterocycle with one or more heterocyclic atoms, wherein R′ can be one of hydrogen, lower alkyl, alkyl-hydroxyl, or can form a closed 3-6 membered heterocycle with one or more heterocyclic atoms, branched alkyl, branched alkyl hydroxyl, where each R′ is independent in case there is more than one

R₂ can be one of hydrogen, alkyl, branched alkyl, phenyl, substituted phenyl, halogen, alkylamino, alkyloxo, CF₃, sulfonamido, substituted sulfonamido, alkyoxy, thioalkyl, sulfonate, sulfonate ester, phosphate, phosphate ester, phosphonate, phosphonate ester, carboxo, amido, ureido, substituted carboxo, substituted amido, substituted ureido, or 3-6 membered heterocycle with one or more hetrocyclic atoms, with the further proviso that either one or two substituents R₂ can be present in the ring, and if more than one substituent R₂ are present, each of the substituents can be the same or different;

R₃ can be one of hydrogen, alkyl, branched alkyl, alkoxy, halogen, CF₃, cyano, substituted alkyl, hydroxyl, alklylhydroxyl, thiol, alkylthiol, thioalkyl, amino, or aminoalkyl; and

n is an integer that can have value between 1 and 5, with the further proviso that if n≧2, then each group R₃ is independent of the other groups R₃.

Some specific, but non-limiting examples of the above-described compounds A that can be used include the compounds described by structures I, II and III shown below:

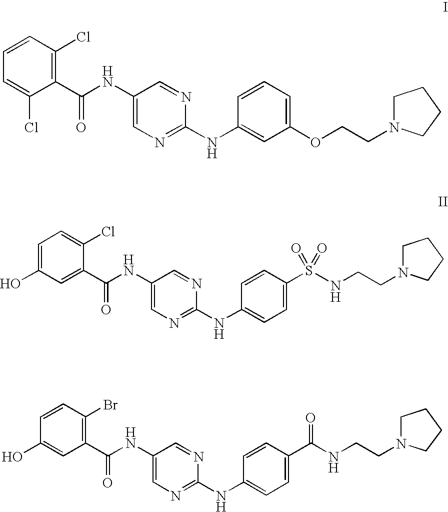

According to another embodiment of the invention, benzotriazine-derived compounds having the structure B, or pharmaceutically acceptable salts, hydrates, solvates, crystal forms, N-oxides, and individuals diastereomners thereof, are provided for treatment of various ocular diseases, disorders, and pathologies.

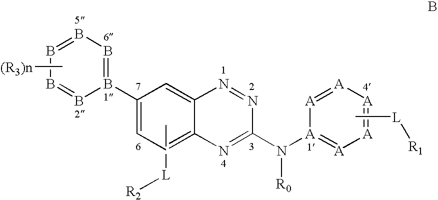

In the structure B, each of A can be independently selected from a group consisting of (CH)₀₋₁, N, NH, O, S, and a part of a ring fusion to form a second ring, where the second ring is an aromatic, a heteroaromatic, a bicyclic aromatic, a bicyclic aromatic heterocyclic ring, or a bicyclic with only the first ring being aromatic or heteroaromatic;

each of B can be independently selected from a group consisting of (CH)₀₋₁, N, NH, O, S, and a part of a ring fusion to form a second ring, where the second ring is an aromatic, a heteroaromatic, a bicyclic aromatic, a bicyclic aromatic heterocyclic ring, or a bicyclic with only the first ring being aromatic or heteroaromatic, with the further proviso that if each B is (CH)₀, R₃ is bonded directly to the adjacent ring.

R₀ can be selected from a group consisting of H and lower alkyl;

L can be selected from a group consisting of a bond, and a substituted or unsubstituted alkyl, alkenyl, or alkynyl linking moiety;

R₁ can be selected from a group consisting of C(R′)₃, OR′, N(R′)₂, NR′C(O)R′, NR′C(O)O(R′), NR′C(O)N(R′)₂, SR′, C(O)(O)R′, C(O)R′, C(O)N(R′)₂, SO₃R′, OSO₂R′, SO₂R′, SOR′, S(O)N(R′)₂, OS(O)(O)N(R′)₂, S(O)(O)N(R′)₂, S(O)N(R′)₂, PO₄R′, OPO₂R′, PO₃R′, PO₂R′, and a 3-6 membered heterocycle with one or more heterocyclic atoms with each heteroatom independently being capable of carrying any R′ group on it, wherein R′ is selected from a group consisting of hydrogen, lower an alkyl, a substituted alkyl, an alkyl-hydroxyl, a substituted alkyl-hydroxyl, a thiol-alkyl, a thiol-substituted alkyl, an alkyl-thiol, a substituted alkyl-thiol, an aminoalkyl, an amino-substituted alkyl, an alkylamino, a substituted alkyl-amino, a branched alkyl, a branched substituted alkyl, a branched alkyl hydroxyl, a branched substituted alkyl hydroxyl, a branched thio-alkyl, a branched thio-substituted alkyl, a branched alkyl-thiol, a branched substituted alkyl-thiol, a branched aminoalkyl, a branched amino-substituted alkyl, a branched alkylamino, a branched substituted alkyl-amino, and a closed 3-6 membered carbocycle or heterocycle, wherein a substitutent in any of said substituted alkyls includes said closed 3-6 membered carbocycle or heterocycle, with the further proviso that each heteroatom in the 3-6 membered heterocycle capable of carrying any R′ group on it, with the further proviso that the substitution in any of said substituted alkyls includes any R′ group connected to said alkyls via an atom other than carbon or via carbon, and wherein each R′ is independent in case there is more than one R′;

R₂ is a substitutent situated at position 5, 6 or 8 of the ring, wherein R₂ can be selected from a group consisting of methyl, ethyl, n-propyl, iso-propyl, n-butyl, iso-butyl, tert-butyl, iso-pentyl, phenyl, substituted phenyl, halogen, branched or unbranched alkylamino, branched or unbranched aminoalkyl, branched or unbranched alkyloxo, branched or unbranched oxyalkyl, branched or unbranched thioalkyl, branched or unbranched alkylthiol, CF₃, sulfonamido, substituted sulfonamido, sulfonate, sulfonate ester, phosphate, phosphate ester, phosphonate, phosphonate ester, carboxo, amido, ureido, substituted carboxo, substituted amido, substituted ureido, or a 3-6 membered carbocycle or heterocycle attached to positions 5, 6 or 8 directly or through group L, each heteroatom independently being capable of carrying any group R₂, with the further proviso that either one, two or three substituents R₂ are present in the ring, each of the substituents R₂ being the same or different;

R₃ can be selected from a group consisting of hydrogen, alkyl, alkoxy, halogen, CF₃, cyano, substituted alkyl, or hydroxyl, aryl, substituted aryl, heteroaryl, substituted heteroaryl, heterocycle, C(R″)₃, OR″, N(R″)₂, NR″C(O)R″, NR″C(O)NR″, R″, C(O)(O)R″, OC(O)R″, C(O)N(R″)₂, C(O), C(O)R″, OC(O)N(R″)₂, SO₃R″, OSO₂R″, SO₂R″, SOR″, PO₄R″, OPO₂R″, PO₃R″, PO₂R″, wherein R″ is hydrogen, aryl, substituted aryl, heteroaryl, substituted heteroaryl, lower alkyl, branched lower alkyl, alkyl-hydroxyl, branched alkyl-hydroxyl, amino-alkyl, branched amino-alkyl, alkyl-amino, branched alkyl-amino, thiol-alkyl, branched thiol-alkyl, alkyl-thiol, branched thiol-alkyl, or may form a closed 3-6 membered heterocycle with one or more heterocyclic atoms, branched alkyl, branched alkyl hydroxyl, where each R″ is independent in case there is more than one R″;

n is an integer having the value between 1 and 5, with the further proviso that if n≧2, then each group R₃ is independent of the other groups R₃;

with the further proviso that if each A is (CH)₀, L is a bond;

with the further proviso that if each B is (CH)₀, R₃ is any substitutent described above, other than hydrogen, bonded directly to the position 7 of the adjacent ring;

and pharmaceutically acceptable salts, hydrates, solvates, crystal forms, N-oxides, and individuals diastereomners thereof.

Some exemplary compounds described by structure B that can be used include, but are not limited to, compounds (IV) through (XX) shown below:

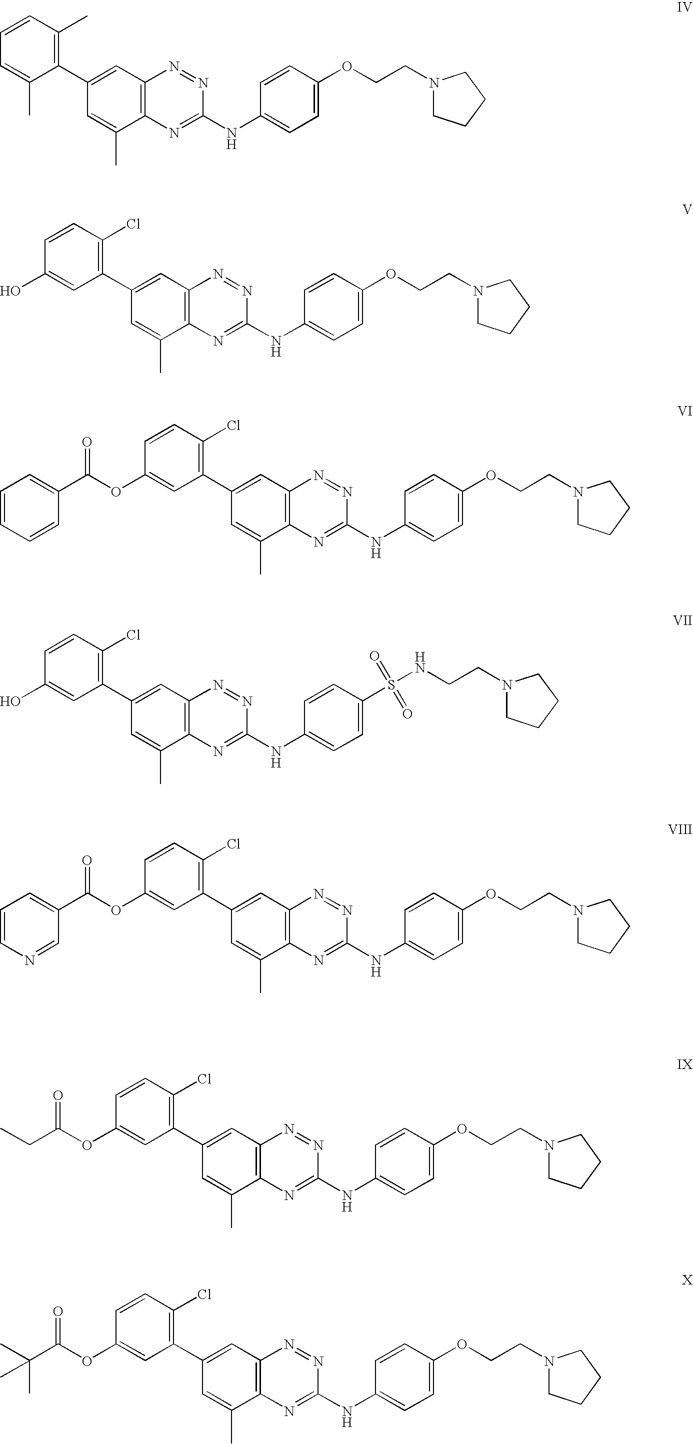

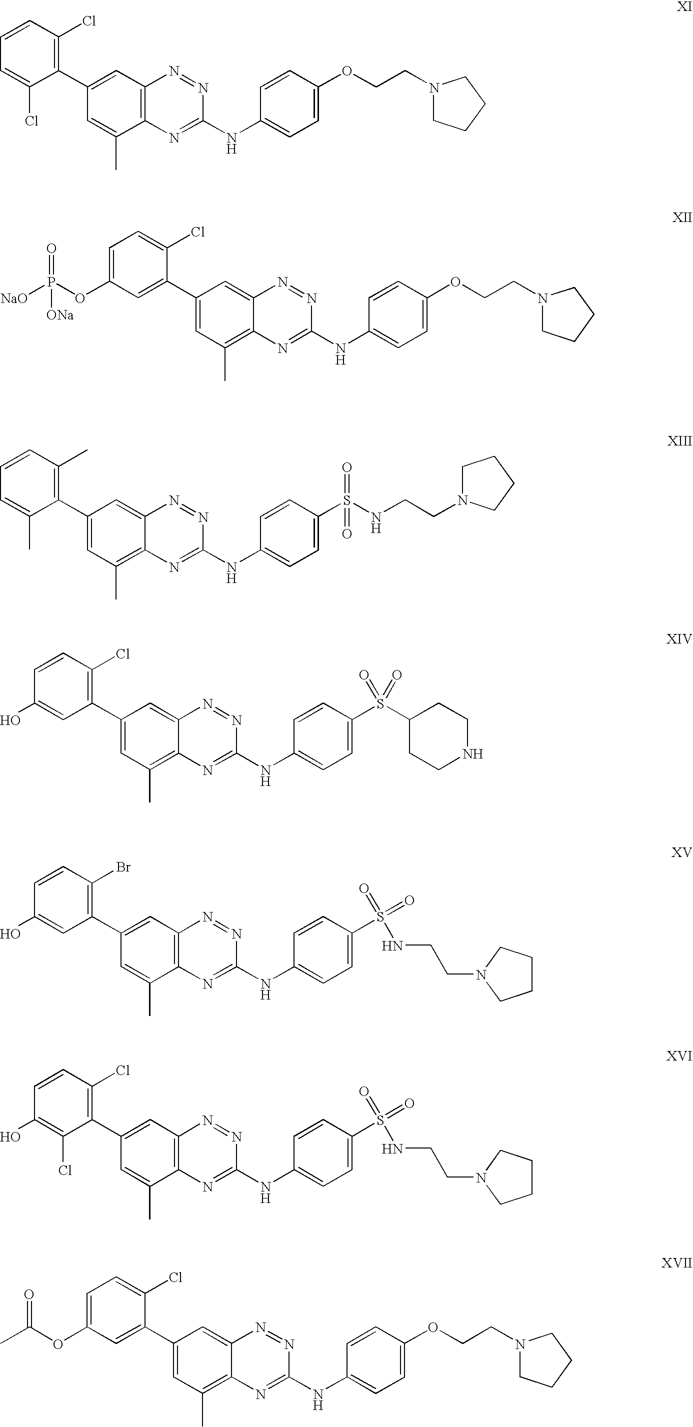

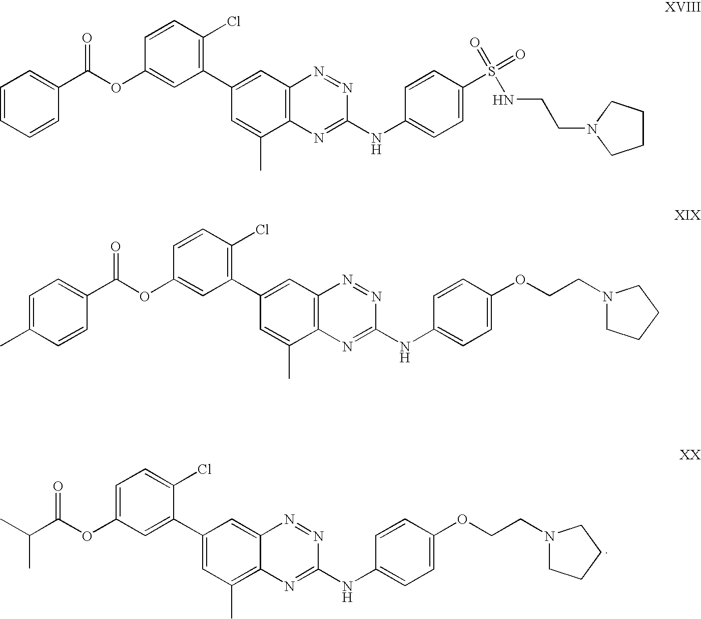

According to embodiments of the present invention, methods for treating an ophthalmological condition in a subject are provided, including administering to a subject in need of such treatment a therapeutically effective amount of a composition of the present invention, thereby treating the condition.

The administration of the composition is designed to treat the specific ophthalmological diseases, pathologies, and disorders, or to reverse the disease, or to reduce the negative effects of the disease, or to reduce the risk of progression of the disease. The non-limiting examples of the diseases, pathologies, and disorders that can be treated include age-related macular degeneration (AMD), dry AMD, diabetic retinopathy, diabetic macular edema, cancer, and glaucoma. Some compositions of the invention can be used for treatment of some ophthalmological diseases, pathologies, and disorders, but not for the treatment of other such diseases, pathologies, and disorders. For example, some compositions are suitable for the treatment of AMD, but not suitable for the treatment of glaucoma, and vice versa. Those having ordinary skill in the art can determine which compounds are or are not suitable for the treatment of particular ophthalmological diseases, pathologies, and disorders.

A number of immunological factors may have been implicated in age-related macular degeneration (AMD) and other eye diseases. It is possible that the presence of immune cells and complement in drusen deposits formed in the macula preceding AMD can further activate inflammatory pathways which contribute to the etiology of the disease. One such pathway may be the recruitment and activation of macrophages which further aggravate inflammation in the eye and may contribute to choroidal neovascularization. A drug or prodrug of this present invention may have immunoregulatory properties upon administration that may be useful in the treatment of diseases where an imbalance in the immune response is present, by having an effect in one or more of the arms of the immune response. The effect can be directly to immune cells like; MHC type I and II, macrophages, T cells, B cells, mast cells, etc. or by altering, enhancing or decreasing specific cytokines or chemokines in a human individual upon administration.

To administer the compositions according to embodiments of the present invention, the compositions of are formulated as eye drops, solutions, suspensions, emulsions, gels, or ointments containing a therapeutically effective amount of the active compound. Typical methods of administration of the compositions described herein include topical delivery, delivery to the back of the eye, intravitreal, or periocular administration. Those having ordinary skill in the art can determine the dosage and the treatment regimen that is suitable for a specific patient. As one non-limiting example, the composition formulated as eye drops can be administered as frequently as from 1 to 4 times a day or as infrequently as 1 to 4 times a week.

The drugs included in the formulations of the present invention may be lipophilic and may be inhibitors of various kinases. Non-limiting examples of kinases that may be inhibited include a Janus family kinases (Jak), Src family kinase, VEGF receptor family kinases, PDGF receptor family kinases, an Eph receptor family kinase, and an FGF receptor family kinases.

Other non-limiting examples of kinases that may be inhibited include, Casein kinases (CK2), CK2, CK2 alpha, CK2 beta, human CK2 (alpha subunit), human CK2 (beta subunit), human CK2 (holo enzyme complex), Zea mays CK2, Akt/PKB: Akt, Akt1, Akt1 (inactive), Akt2, Akt3, PKB, PKB alpha, PKB alpha (inactive), PKB beta, PKB gamma, MAP kinase pathway: ERK, ERK1, ERK2, JNK2, JNK2alpha, MAP2K1, MAPK1, MAPK3, MAPKK1, MAPKK6, MEK1, MKK1, MKK6, p38, p38 (inactive), p38a/SAPK2a, SAPK1, SAPK2, including Ras and Raf and other kinases in these and related pathways, and various other kinases, as in ABL, ARK5, Aurora-A, Aurora-B, Aurora-C, BRK, CaMKII, CDK1/B, CDK2/A, CDK2/E, CDK3/E, CDK4/D1, CDK5/p35NCK, CDK6/D1, CDK7/H/MAT1, CDK9/CycT, CHK1, CHK2, c-KIT, c-MET, COT, CSK, DAPK1, EGFR, EPHA, EPHB, ERBB2, ERBB4, FAK, FGF-R, FGR, FLK1, FLT3, GSK3 beta, HER2, IGF1-R, IKK beta, INS-R, ITK, JAK2, JAK3, JNK3, KDR, KIT, LCK, LYN, MET, MST4, MUSK, NEK2, NEK6, NLK, PAK, PDGFR, PDK1, PIM, PKC alpha, PKC beta, PKC delta, PKC epsilon, PKC eta, PKC gamma, PKC iota, PKC mu, PKG, PLK1, PRK1, PRKX, PTK2, RET, ROCK2, S6K4, SAK, SGK, SRC, SYK, thymidine kinase TK1, TIE2, VEGFR1, VEGFR2, VEGFR3, ZAP70, or any other kinases related to mediating or involved with vascular leakage or angiogenesis, or inflammatory response.

In addition to the above-described active compounds and pharmaceutically acceptable carriers, the compositions of the present invention optionally further include antiviral agents, antibiotics, intraocular pressure reducing compositions, wetting agents, cataract prevention agents, RNAi molecules, antisense molecules, peptides, polynucleotides, proteins, small molecule compounds, VEGF inhibitors, anti-inflammatory agents, oxygen radical scavenger agents, tonicity agents, comfort-enhancing agents, solubilizing aids, antioxidants, stabilizing agents, and NO inhibitors.

Various methods can be used to prepare the compositions of the invention. In one embodiment, the drug or prodrug to be used is fully or partially dissolved in the presence or absence of an organic solvent, followed by mixing with an aqueous colloidal suspension containing a polymer base carrier with or without a surface active component. The solvent may be then removed (if used), osmotic agents may be added, and pH may be adjusted to make the composition suitable for administration. The method may also optionally include adding aseptic filling, or sterilization by filtering or autoclaving, or freeze-drying, or spray-drying, or reconstitution of dry formulation before usage, or a combination of such optional steps.

In another embodiment, the drug or prodrug is used may be mixed with an aqueous colloidal suspension containing a polymer base carrier to form a colloidal suspension—for example, a suspension having a mean particle size less than 5 μm, such as less than 1 μm, followed by adding osmotic agents, followed by adjusting the pH to a range suitable for administration. If desired, the method may also optionally include adding aseptic filling, or sterilization by filtering or autoclaving, or freeze-drying, or spray-drying, or reconstitution of dry formulation before usage, or a combination of such optional steps.

The compositions of the present inventions may be formulated as water continuous colloidal suspensions. The lipids included in such suspensions may be surface active. Some non-limiting examples of lipids that may be used in the formulations of the present invention include phospholipids, phosphatidylcholines, cardiolipins, fatty acids, phosphatidylethanolamines, and phosphatides. Such colloidal suspensions may further include a polymer that is capable of forming the suspensions when combined with the drug to be included into the composition, e.g., a lyophilic polymer. Some non-limiting examples of polymers that may be used in formation of such suspensions include cellulose derivatives such as hydroxypropylmethyl cellulose (HPMC), carboxymethyl cellulose (CMC), methyl cellulose (MC), hydroxyethyl cellulose (HEC), amylose and derivatives, amylopectins and derivatives, dextran and derivates, polyvinylpyrrolidone (PVP), polyvinyl alcohol (PVA), and acrylic polymers such as derivatives of poly(acrylic) or poly(methacrylic acid), like HEMA, carbopol (from Noveon or similar polymers) The colloidal suspensions of the present invention may also include surface active components used as wetting/dispersing agents that are well tolerated in the eye. The non-limiting examples of surfactants are primarly non-ionic surfactants, like tyloxapol, polyethylenglycols and derivatives, like PEG400, PEG1500, PEG20000, poloxamer 407, poloxamer 188, tween 80, and polysorbate 20. These surface active components may be used alone or combination with other surface active components or in combination with the lipids and the polymers described above.

These compositions may include one or more preservatives such as benzalkonium chloride, alkyldimethylbenzylammonium chloride, cetrimide, cetylpyridinium chloride, benzododecinium bromide, benzethonium chloride, thiomersal, chlorobutanol, benzyl alcohol, phenoxyethanol, phenylethyl alcohol, sorbic acid, methyl and propyl parabens, chlorhexidine digluconate, or EDTA.

The compositions of the invention may be formulated in a salt form. Pharmaceutically acceptable non-toxic salts include the base addition salts (formed with free carboxyl or other anionic groups) which may be derived from inorganic bases such as, for example, sodium, potassium, ammonium, calcium, or ferric hydroxides, and such organic bases as isopropylamine, trimethylamine, 2-ethylamino-ethanol, histidine, procaine, and the like. Such salts may also be formed as acid addition salts with any free cationic groups and will generally be formed with inorganic acids such as, for example, hydrochloric, sulfuric, or phosphoric acids, or organic acids such as acetic, citric, p-toluenesulfonic, methanesulfonic acid, oxalic, tartaric, mandelic, and the like. Salts of the invention include amine salts formed by the protonation of an amino group with inorganic acids such as hydrochloric acid, hydrobromic acid, hydroiodic acid, sulfuric acid, phosphoric acid, and the like. Salts of the invention also include amine salts formed by the protonation of an amino group with suitable organic acids, such as p-toluenesulfonic acid, acetic acid, and the like. Additional excipients which are contemplated for use in the practice of the present invention are those available to those of ordinary skill in the art, for example, those found in the United States Pharmacopoeia Vol. XXII and National Formulary Vol. XVII, U.S. Pharmacopoeia Convention, Inc., Rockville, Md. (1989), the relevant contents of which is incorporated herein by reference. In addition, polymorphs of the compounds described herein are included in the present invention.

In another embodiment of the present invention, a method for treating an ophthalmological condition in a subject is provided including administering to a subject in need of such treatment a therapeutically effective amount of a composition of the present invention by delivery of the composition to the back of an eye. For such delivery, the formulation can be in the form of eye drops. The method may further include administration of a kinase inhibitor, such as an inhibitor of the Src family kinases, the VEGF receptor family kinases, the PDGF receptor family kinases, the Eph receptor family kinases, or the FGF receptor family kinases.

According to another embodiment of the present invention, a compound suitable for delivery to the eye can be identified. To make such identification, a compound is administered to the eye by eye drop administration, and the distribution of the compound in the eye is observed following eye drop administration, thereby identifying a compound suitable for delivery to the eye with the proviso that a candidate compound is not a steroidal molecule. A compound used in such a method has a polar surface area not exceeding about 150 Å2, such as less than about 120 Å2, for example, not exceeding about 100 Å2. The compound further has a water solubility of less than about 0.1 mg/mL at a pH range of 4-8, such as less than about 0.05 mg/mL at a pH range of 4-8, for example, less than about 0.01 mg/mL at a pH-range of 4-8. The compound additionally has a cLogD of at least about 0.5 at pH of 7.4, such as at least about 1, for example, at least 2. The compound further has a molecular weight not exceeding about 1,000 Daltons, such as not exceeding about 900 Daltons, for example, not exceeding about 800 Daltons.

According to another embodiment of the present invention, an article of manufacture is provided. The article may comprise a vial, container, tube, flask, dropper, and/or a syringe, containing a composition as described herein for ophthalmic delivery including an active compound and may further include instructions for administration of the composition.

The following examples are provided to further illustrate the advantages and features of the present invention, but are not intended to limit the scope of the invention. Representative results for Ocular efficacy and for demonstration of delivery via pharmacokinetic analysis of the back of the eye tissues of some compounds from the invention, following eye drop delivery of the compounds may be found in the FIGURES

### EXAMPLE 1

### Preparation of Water Continuous Lipid Based Colloidal Suspension Containing Compound (V)

A water continuous lipid based colloidal suspension was prepared by taking 18 mg of Compound (V) in the form of a HCl salt, mixing with 550 mg of dimyristoyl phosphatidylcholine (DMPC), 2412 mg of a 2.9% propylene glycol, and homogenizing using a sonicator probe in a temperature controlled bath. The pH was adjusted to 5-6 using 35 μL of a 0.1 N NaOH, and the composition was further sonicated to ensure homogeneity. The resulting formulation was sterile filtered through a 0.22 μm PVDF syringe filter.

Alternatively, the drug may be homogenized using high pressure homogenization. If desired, the drug may be pre-dissolved with the lipid prior to homogenization in water with the aid of an organic solvent such ethanol or chloroform. If desired, the resulting formulation may also be autoclaved to achieve sterility in the final container. If desired, preservatives, such as benzalkonium chloride, may be added.

### EXAMPLE 2

### Preparation of Water Continuous Lipid Based Colloidal Suspension Containing Compound (XI)

A water continuous lipid based colloidal suspension was prepared by taking 37.6 mg of Compound (XI) in the form of an HCl salt, mixing with 550 mg of DMPC, 2412 mg of a 2.9% propylene glycol, and homogenization using a sonicator probe in a temperature controlled bath. The pH was adjusted to 5-6 using 15 μL of a 50 mg/mL sodium oleate in de-ionized water, and the suspension further sonicated to ensure homogeneity. The resulting formulation was sterile filtered through a 0.22 μm PVDF syringe filter.

Alternatively, the drug may be homogenized using high pressure homogenization. If desired, optionally the drug may be pre-dissolved with the lipid prior to homogenization in water with the aid of an organic solvent such ethanol or chloroform. If desired, the resulting formulation may also be autoclaved to achieve sterility in the final container. If desired, optionally preservatives, such as benzalkonium chloride, may be added.

### EXAMPLE 3

### Pharmacokinetic Studies of Compound (XI) in Dutch-Belted Rabbits After Topical Administration

A formulation was prepared as in Example 1 but using Compound (XI) instead of Compound (V). Compound (XI) was administered as eyedrops (1% API, 50 μL) BID for 3 days. On day 3 following a single dose, rabbits were sacrificed, enucleated and various ocular tissues (retina, choroid, cornea, etc) collected. Concentrations in the tissues were measured using LC/MS/MS, following tissue homogenization and acetonitrile precipitation. PK data analysis was conducted using WINNONLIN program. Concentrations of compound V in the choroid were similar between the 2 formulations (at the μM level). Half-life was long at approximately 8 hours.

### EXAMPLE 4

### Pharmacokinetic Studies of Compound (V) in Dutch-Belted Rabbits After Topical Administration

A formulation containing Compound (V) prepared as described in Example 1 was used in this experiment. 50 μL of Compound (V) (QD for one day) was administered topically to rabbits at 0.5% dose. Ocular tissues such as choroid, retina, sclera and cornea were collected and concentrations measured. Choroidal concentrations were 4 fold higher than retinal concentration. Half-life was about two times longer.

### EXAMPLE 5

### Preparation of Water Continuous Lipid Based Colloidal Suspension Containing Compound (VI)

A water continuous lipid based colloidal suspension containing the active at 1% dose was prepared by taking 13 mg of Compound (VI), as a free base, homogenizing at about 50-60° C. in the presence of 830 mg of a solution containing 0.125% HPMC 4KM in 5% dextrose and 36 μL of a 1 N HCl, until a clear translucid colloidal sol was obtained. Then 205 mg of an 18% lipid vesicle of saturated soy phosphatidylcholine (PL90H) in 2.9% propylene glycol was added as a stabilizer to reduce colloid flocculation. The sample was sonicated and pH was adjusted with the addition of 24 μL of a 1 N NaOH to a suitable physiological pH between 4.5 and 6. The sample was further homogenized by sonication or high pressure homogenization and filtered through a 0.45 μm PVDF syringe filter. Osmolality was 319 mmolal.

Optionally, the above described formulation can be obtained without using surfactant (i.e., a phospholipid). In such case, the appropriate charge on the particle may need to be maintained by introducing a counterion that will adsorb on the surface of the particle and maintained there, with an adequate pH to reduce flocculation.

### EXAMPLE 6

### Preparation of Water Continuous Lipid Based Colloidal Suspension Containing Compound (VI)

A water continuous lipid based colloidal suspension containing the active at 0.5% dose was prepared by taking 13 mg of Compound (VI) as a free base, homogenizing at about 50-60° C. in the presence of 1620 mg of a solution containing 0.125% HPMC 4KM in 5% dextrose and 36 μL of a 1 N HCl, until a clear translucid colloidal sol was obtained. Then 384 mg of an 18% lipid vesicle of saturated soy phosphatidylcholine (PL90H) in 2.9% propylene glycol was added as a stabilizer to reduce colloid flocculation. The sample was sonicated and pH was adjusted with the addition of 24 μL of a 1 N NaOH to a suitable physiological pH between 4.5 and 6. The sample was further homogenized by sonication or high pressure homogenization and filtered through a 0.45 μm PVDF syringe filter. Osmolality was 293 mmolal.

Optionally, the above described formulation may be obtained without using surfactant (i.e., a phospholipid). In such case, the appropriate charge on the particle may need to be maintained by introducing a counterion that will adsorb on the surface of the particle and maintained there, with an adequate pH to reduce flocculation.

### EXAMPLE 7

### Preparation of Water Continuous Lipid Based Colloidal Suspension Containing Compound (VI)

A water continuous lipid based colloidal suspension containing the active at 0.2% dose was achieved by taking 382 mg of formulation containing 0.5% of compound (VI) and diluting to a final weight of 982 mg with 0.125% HPMC 4KM in 5% dextrose. The resulting mixture was sonicated mildly to ensure homogeneity. The pH was adjusted to give a final pH of 4.8. The sample was filtered through a 0.45 μm PVDF syringe filter. Osmolality was 282 mmolal.

Optionally, the above described formulation may be obtained without using surfactant (i.e., a phospholipid). In such case, the appropriate charge on the particle may need to be maintained by introducing a counterion that will adsorb on the surface of the particle and maintained in this manner, with an adequate pH to reduce flocculation.

### EXAMPLE 8

### Pharmacokinetic Studies of Compound (VI) in Lone Evans Rat Pups After Topical Administration

Formulations prepared as described in Example 5 were used. Rat pups were administered single 10 μL eyedrops of 0.2, 0.5 or 1% Compound (VI) dose. Eye tissues were collected at various time points for Compound (V) analysis using LC/MS/MS. The mean AUC in the choroid was linear between 0.2 and 1% dose, however, in the retina the concentrations appear to be non-linear. Half-life of Compound (V) ranged from 5 to 8 hours in the choroids.

### EXAMPLE 9

### Preparation of Water Continuous Lipid Based Colloidal Suspension Containing Compound (X)

A water continuous colloidal suspension containing the active at 0.5% dose was prepared by using 51 mg of Compound (X) as the mesylate salt, was homogenizing at about 50-60° C. in the presence of 7.06 g of a solution containing 0.25% HPMC 4KM in 5% dextrose until a clear translucid colloidal sol was obtained. The pH was adjusted by the addition of 1 N NaOH to obtain a final pH measured at 4.7. The sample was further homogenized by sonication or high pressure homogenization and filtered through a 0.45 μm PVDF syringe filter. Final osmolality was 285 mmolal.

### EXAMPLE 10

### Preparation of Water Continuous Lipid Based Colloidal Suspension Containing Compound (X) Using Lipid Surfactant

A water continuous colloidal suspension containing the active at 0.5% dose was obtained by taking 44 mg of Compound (X), homogenizing at about 50-60° C. in the presence of 4.2 g of a solution of dextrose with 1.38 g of a solution containing 0.5% HPMC 4KM in 5% dextrose and 23.8 μL of a 5 N HCl solution, until a clear translucid colloidal sol was obtained. Then 1.23 g of an 18% lipid vesicle of saturated soy phosphatidylcholine (PL90H) in 2.9% propylene glycol was added as a stabilizer to reduce colloid flocculation. The sample was sonicated and pH adjusted with the addition of 50 μL of a 1 N NaOH to a pH between 4.5 and 6. The sample was further homogenized by sonication or high pressure homogenization and filtered through a 0.45 μm PVDF syringe filter. Osmolality was 297 mmolal.

### EXAMPLE 11

### Preparation of Water Continuous Lipid Based Colloidal Suspension Containing Compound (VIII) Using No Surfactant

A water continuous colloidal suspension containing the active at 0.5% dose was obtained by taking 35.6 mg of Compound (VIII) in a free base form and homogenizing at about 50-60° C. in the presence of 5.04 g of a solution containing 0.5% HPMC 4KM in 5% dextrose until a clear translucid colloidal sol was obtained. The actual final pH was 6.68. The sample was filtered through a 0.45 μm PVDF syringe filter. Osmolality was 322 mmolal.

### EXAMPLE 12

### Pharmacokinetic Studies of Compounds (X) and (VIII) in Dutch-Belted Rabbits After Topical Administration

Formulations prepared as described in Example 9 and 11 were used. Compounds (X) and (VIII) were administered as eyedrops (50 μL) either as QD for three days or BID for three days as the dose regimen. Compound (VIII) concentrations in the choroid and retina were not detectable. Concentrations of Compound (V) in the choroids following Compound (X) administration were very reproducible (380-513 nM) and half-life ranged from 7 to 14 hours.

The retinal concentrations varied depending on the formulation used. The cLogD at pH of 7.4 for Compound (VIII) is 0.14 while for Compound (X) is 3.54. No measurable amount of API (Compound (V)) was recovered from the retina and the choroid when the prodrug Compound (VIII) was delivered topically to the eye following the same dosing regimens as the one shown above for Compound (X)

### EXAMPLE 13

### Preparation of Water Continuous Lipid Based Colloidal Suspension Containing Compound (VI) Using No Surfactant

Preparation of a water continuous colloidal suspension containing the active at 1% dose was achieved by taking 50 mg of Compound (VI), followed by homogenizing at about 50-60° C. in the presence of 4.06 g of a solution containing 0.5% HPMC 4KM in 5% mannitol, 90 μL of 1 N HCl and 3 mL of ethanol until a clear translucid colloidal was obtained. Finally, the pH was adjusted by the addition of 112 μL of 0.1 N NaOH to a suitable physiological pH between 4.5 and 6. The ethanol was evaporated and the solution frozen, followed by freeze-drying, then reconstitution with 3.7 g of DI water and filteration through a 0.45 μm PVDF syringe filter.

### EXAMPLE 14

### Pharmacokinetic Studies of Compound (VI) in Dutch-Belted Rabbits After Topical Administration

A formulation prepared as described in example 13 was used. Compound (VI) was administered topically (50 μL) to rabbits either as BID for three days or QD for three days dose regimen (1% dose). Concentrations detected in the tissues in the back of the eye were high (in the IM range) and linear between the 2 dose regimens described.

### EXAMPLE 15

### Preparation of Water Continuous Lipid Based Colloidal Suspension Containing Compound (IV)

A water continuous lipid base colloidal suspension containing 51.1 mg of Compound (IV) as an HCl salt was mixed with 830 mg of phosphatidylcholines (PL90G from American Lecithin), and dissolved in 2.5 mL of ethanol, followed by concentration to dryness (under high vacuum), resuspending using 7.1 g of a 2.9% w/v propylene glycol (USP)+12 μL of 1 N NaOH, homogenization using a sonicator probe, followed by the addition of 0.3 mL of a 0.9% NaCl and pH adjustment to 5.5 using 0.1 N HCl. The resulting formulation was sterile filtered through a 0.22 μm PVDF syringe filter. Osmolality was 314 mMolal.

### EXAMPLE 16

### Preparation of Water Continuous Lipid Based Colloidal Suspension Containing Compound (XI)

A water continuous lipid base colloidal suspension containing 51.8 mg of Compound (XI) as a HCl salt was mixed with 810 mg of phosphatidylcholines (PL90G from American Lecithin) and dissolved in 2.5 mL of ethanol, followed by evaporation to dryness (under high vacuum), resuspension with 7.1 g of a 2.9% w/v propylene glycol (USP)+12 μL of 1 N NaOH, homogenization using a sonicator probe, addition of 0.3 mL of a 0.9% NaCl, followed by a final pH adjustment to 5.5 with 0.1N HCl. The resulting formulation was sterile filtered through a 0.22 μm PVDF syringe filter. Osmolality was 320 mMolal.

### EXAMPLE 17

### Preparation of Water Continuous Lipid Based Colloidal Suspension Containing Compound (V)

A water continuous lipid base colloidal suspension containing 50.6 mg of Compound (V) as an HCl salt was mixed with 1516 mg of phosphatidylcholines (PL90G from American Lecithin) and dissolved in 2.5 mL of ethanol, followed by evaporated to dryness (under high vacuum), re-suspension with 6.4 g of a 2.9% w/v propylene glycol (USP)+12 μL of 1 N NaOH, homogenization using a sonicator probe, followed by the addition of 0.3 mL of a 0.9% NaCl, and a final pH was adjustment to 5.5 with 0.1 N HCl. The resulting formulation was sterile filtered through a 0.22 μm PVDF syringe filter. Osmolality was 330 mMolal.

### EXAMPLE 18

### Preparation of Water Continuous Lipid Based Colloidal Suspension Containing Compound (VII)

A water continuous lipid base colloidal suspension 51.2 mg of Compound (VII) as a HCl salt was mixed with 1521 mg of phosphatidylcholines (PL90G from American Lecithin) and dissolved in 2.5 mL of ethanol, followed by evaporation to dryness (under high vacuum), resuspension with 6.4 g of a 2.9% w/v propylene glycol (USP)+12 μL of 1 N NaOH, homogenization using a sonicator probe, and 0.3 mL of a 0.9% NaCl, and a final pH adjustment to 5.5 with 0.1 N HCl. The resulting formulation was sterile filtered through a 0.22 μm PVDF syringe filter. Osmolality was 334 mMolal.

### EXAMPLE 19

### Pharmacokinetic Studies of Compounds (VII), V), (XI), and (IV) in Dutch-Belted Rabbits After Topical Administration

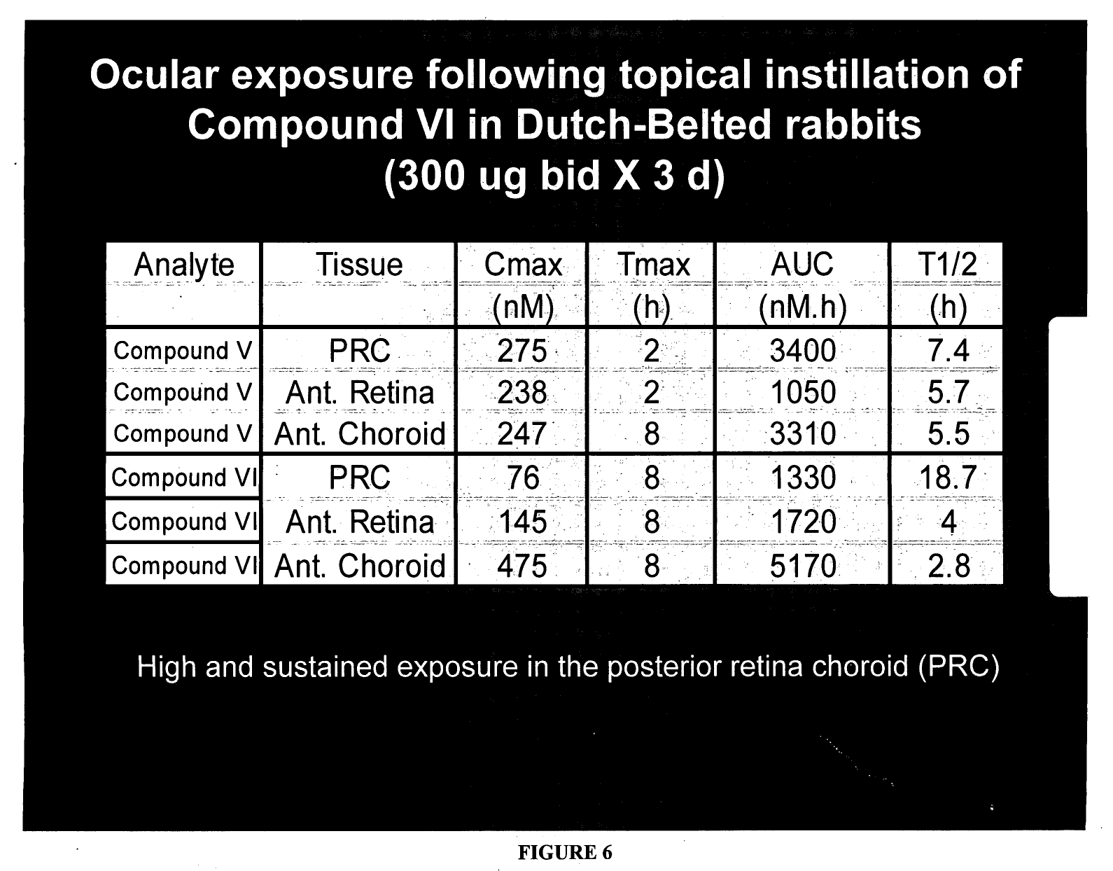

Formulations were prepared as described in Examples 15-18 were used. Compounds (IV), (XI), (V), and (VII) were administered topically (50 μL/eye) at 0.5% dose (BID) for 5 days to rabbit eyes. Ocular exposure at steady state was determined at 1, 7 and 24 h. Cmax in the choroid for 598 and 572 ranged from 208 to 290 ng/ml. The results are summarized in FIG. 6.

### EXAMPLE 20

### Preparation of Water Continuous Lipid Based Colloidal Suspension Containing Compound (VI) Using No Surfactant

A water continuous colloidal suspension containing the active at 1% dose was prepared by taking 50 mg of Compound (VI) as a free base and homogenizing at about 50-60° C. in the presence of 4.06 g of a solution containing 0.5% HPMC 4KM in 5% mannitol, 90 μL of 1 N HCl and 3 mL of ethanol until a clear translucid colloidal was obtained. Finally the pH was adjusted with the addition of 112 μL of 0.1 N NaOH to a obtain a suitable value between 4.5 and 6. The ethanol was evaporated, and the solution was frozen, followed by freeze-drying, then reconstituting with 3.7 g of de-ionizedwater and filtering through a 0.45 μm PVDF syringe filter.

### EXAMPLE 21

### Preparation of Water Continuous Lipid Based Colloidal Suspension Containing Compound (V) Using Lipid Surfactant

A water continuous lipid base colloidal suspension containing 31.16 mg of Compound (V) as a HCl salt was mixed with 970 mg of phosphatidylcholines (PL90G from American Lecithin) and dissolved in 2 mL of ethanol, followed by evaporation to dryness (under high vacuum), resuspension with 2.7 g of a 2.9% w/v propylene glycol (USP)+12 μL of 1 N NaOH, homogenization using a sonicator probe, addition of 0.2 mL of a 0.9% NaCl. The final pH was 6.1. The resulting formulation was sterile filtered through a 0.22 μm PVDF syringe filter. Osmolality was 355 mMolal.

### EXAMPLE 22

### Efficacy Studies in an Ocular Model of Retinal Edema Following Eyedrops

Formulations prepared as described in Examples 20 and 21 were used in these studies. Topical eyedrops of Compound (V) (one time or three times a day), or Compound (VI) (single eye drop) were administered to mice. After 1-2 hr, VEGF was injected intravitreally into mouse eyes. An hour later Evans Blue dye was injected intravenously into the tail vein. About 4 hrs later animals were sacrificed, blood was collected and eyes were enucleated. VEGF-induced retinal permeability as measured by albumin leakage in the eye was measured.

Following QD administration of Compound (V), retinal leak was inhibited by 50%, however results were not statistically significant. Following TID dosing of Compound (V), retinal leak was inhibited by ˜80% (p<0.00003). Retinal leak was completely inhibited (100%) following QD dosing of Compound (VI) (p<0.00002).

### EXAMPLE 23

### Preparation of Water Continuous Lipid Based Colloidal Suspension Containing Compound (V) Using Lipid Surfactant

A water continuous lipid base colloidal suspension containing 15.29 mg of Compound (V) as a HCl salt was mixed with 471 mg of phosphatidylcholines (PL90G from American Lecithin) and dissolved in 1 mL of ethanol, followed by evaporation to dryness (under high vacuum), resuspension with 4.5 g of a 2.3% w/v propylene glycol (USP)+40 μL of 0.1 N NaOH, homogenization using a sonicator probe, with a final addition of 0.125 mL of a 0.9% NaCl. The final pH was 5.5. The resulting formulation was sterile filtered through a 0.22 μm PVDF syringe filter. Osmolality was 255 mMolal.

### EXAMPLE 24

### Preparation of a Suspension of Compound (V) in 5% Dextrose

34.70 mg of Compound (V) as an HCl salt was mixed with 3 mg of hydrogenated phosphatydylcholine (PL90H) and suspended in 5% dextrose to a final weight of 3 g. The composition was sonicated for two hours to reduce the particle size in the range of 5-10 μm, and the final pH was adjusted to 5.5 with 1 N NaOH. This suspension was diluted with 5% dextrose to give a final drug concentration of 3 mg of active per mL. The sample was heat sterilized and delivered to rats via eye drop adminstraiondropadministration.

### EXAMPLE 25

### Efficacy Studies in the Delivery by a Water Continuous Drug Delivery System to the Back of the Eye

Formulations described in Examples 23 and 24 were prepared. The first formulation is a water continuous lipid based colloidal system, while the second formulation is a micron sized suspension in water of the same drug.

### EXAMPLE 26

### Preparation of Samples of Compound (VI) for Efficacy Testing to Suppress Choroidal Neovascularization

128 mg of Compound (V) was mixed with 7 g of 26% w/v suspension of phosphatidylcholines (PL90G from American Lecithin) in 2.6% propylene glycol and 360 μL of 1 N HCl, homogenized using a sonicator probe, in a cool bath until translucid. Then 100 μL of a 0.9% NaCl and 138 μL of a 1 N NaOH were added, to adjust pH to 5.65. The resulting formulation was sterile filtered through a 0.245 μm PVDF syringe filter. Osmolality was 372 mMolal.

### EXAMPLE 27

### Preparation of Samples of Compound (VI) for Efficacy Testing to Suppress Choroidal Neovascularization

50 mg of Compound (V) was mixed with 4 g of 18% w/v suspension of phosphatidylcholines (PL90G from American Lecithin) in 2.6% propylene glycol and 136 μL of 1 N HCl, homogenized using a sonicator probe, in a cool bath until translucid. Then 54 μL of a 1 N NaOH was added to adjust pH to 5.8. The resulting formulation was sterile filtered through a 0.245 μm PVDF syringe filter. Osmolality was 443 mMolal.

### EXAMPLE 28

### Preparation of Lipid Vesicles Control Samples

2689 mg of phosphatidylcholines (PL90G from American Lecithin) was homogenized using a sonicator probe (a high pressure homogenizer can be utilized) in a cool bath until translucid, filtered through a 0.45 μm PVDF syringe filter.

### EXAMPLE 29

### Topical Administration of Compound (VI) for Suppressing Choroidal Neovascularization and Retinal Leaks

Formulations prepared as described in Examples 26-28 were used. The Compound (VI) was tested in a model of choroidal angiogenesis in which angiogenesis was induced using laser-induced rupture of the Bruch's membrane of C57BL/6 mice.

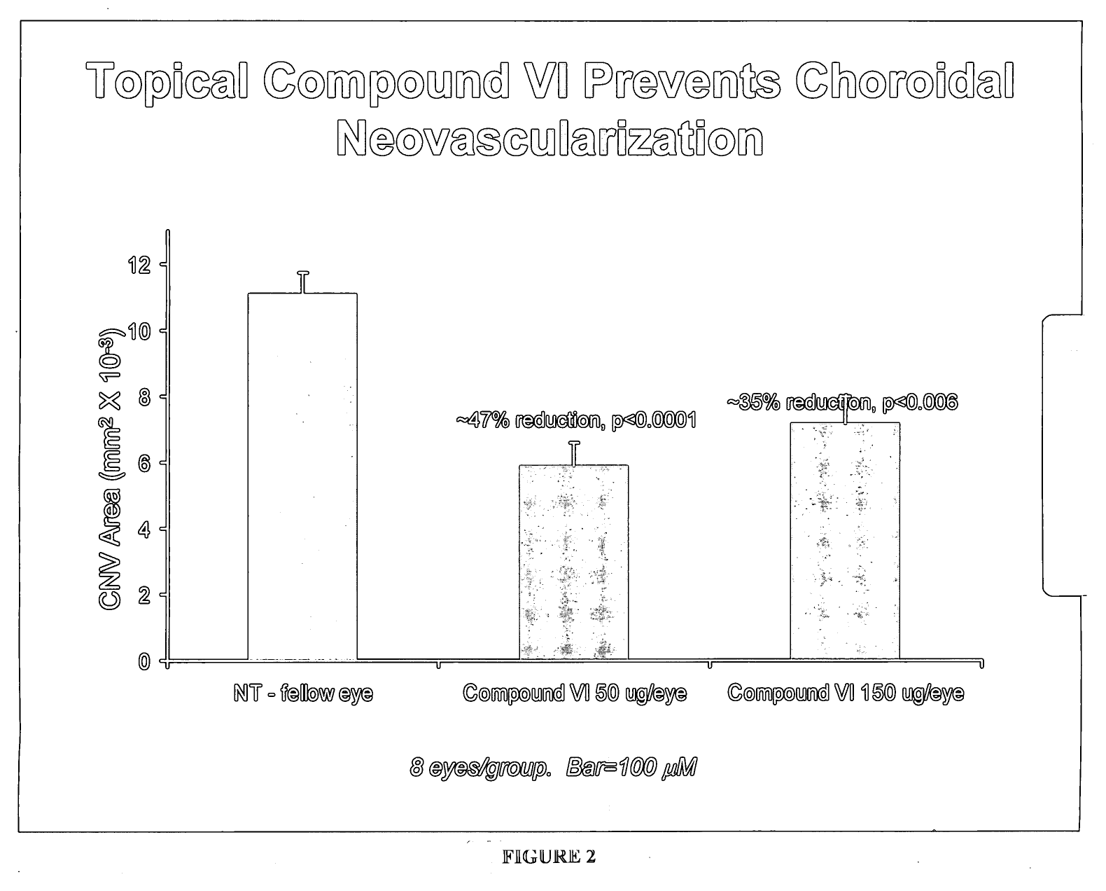

4 to 5 week old female C57BL/6J mice (n=10/group) were delivered three burns of 532 nm diode laser photocoagulation at 9, 12, and 3 o'clock positions of the posterior pole of the retina. After laser burn, mice were treated with vehicle or Compound (V) as indicated. After 2 weeks, mice were perfused with fluorescein-labeled dextran, and choroidal flatmounts were analyzed using image analysis software to recognize fluorescently stained neovascularization and calculate the total area of neovascularization per retina. The results showed that Compound (VI) dosed at 50 μg per eye exhibited approximately 47% reduction (p<0.0001) and dosed at 150 μg per eye exhibited a reduction of approximately 35% (p<0.006) compared to a control sample. The results are summarized in FIG. 2.

### EXAMPLE 30

### Study of Exposure to Compounds (V) and (VI) Following Bilateral Topical Instillation of Compound (VI) in Rabbit, Min-Pig and Dog

Composition examples 30-A through 30-F were prepared as described below and evaluated. Preparation of formulation 30-A (1% Compound (VI) in 5% PL90H/0.2% HPMCdextrose)

181.82 mg of Compound (VI) was dispersed using 6.7 g of a 0.5% HPMC (SIGMA, 40-60 cps) in sterile water for irrigation (SWFI) and 102 μL of a 5 N HCl, while mixing and heating (˜50° C.) until translucid. Then, 8.2 g of a 9% hydrogenated soy lecithin (PL90H—American Lecithin Co) dispersion in water and 60 μL of a 2 N NaOH solution were added to adjust pH between 5.3-6. The compositions was homogenized using sonicator probe (model GE-130), then osmolality was adjusted to approximately 230-240 mOsm with 491 mg of Dextrose (EP/BP/USP grade, Fisher Scientific). The product was filtered through a 0.45 μm PVDF syringe filter (Millipore), followed by filtration using a 0.22 μm PVDF syringe filter (Millipore).

### Preparation of Formulation 30-B (1% Compound (V) in 0.2% Poloxamer 407/0.3% HPMC/3.5% Dextrose)

107.09 mg of Compound (VI) was dispersed using 5.89 g of a 0.5% HPMC (40-60 cps) in sterile water for irrigation (SWFI) and 54.4 μL of a 5 N HCl, while mixing and heating (˜50° C.) until clear. Then 1.6 g of a 1% Lutrol F127 (BASF) solution and 109 μL of a 2 N NaOH solution was added to adjust pH between 5.3-6. The composition was homogenized using sonicator probe (model GE-130), then osmolality was adjusted to approximately 283 mOsm with 261 mg of Dextrose (EP/BP/USP grade, Fisher Scientific). The product was filtered through a 0.22 μm PVDF syringe filter (Millipore).

### Preparation of Formulation 30-C (0.5% Compound (V) in 5% DMPC/0.2% HPMC/3.7% Dextrose)

49.5 mg of Compound (VI) was dispersed using 3.5 g of a 0.5% HPMC E50 in SWFI and 27.2 μL of a 5 N HCl, while mixing and heating (˜50° C.) until clear. Then 16 μL of a 2 N NaOH was added while mixing followed by adding 4.3 g of a 9% DMPC dispersion and 38.4 μL of a 2 N NaOH solution to adjust pH between 5.3-6. The composition was then homogenized using sonicator, then osmolality was adjusted to approximately 230-240 with 294 mg of dextrose. The final product was filtered through a 0.451 μm filter.

### Preparation of Formulation 30-D (1% Compound (V) in 6% DMPC/0.13% HPMC/3.6% Dextrose)

50.52 mg of Compound (VI) was dispersed using 1.1 g of a 0.5% HPMC E50 in SWFI and 27.2 μL of a 5 N HCl, while mixing and heating (Q50° C.) until clear. Then 2.67 g of a 9% DMPC dispersion and 54.4 μL of a 2 N NaOH solution were added to adjust pH between 5.3-6. The composition was homogenizes using sonicator, then osmolality was adjusted to approximately 230-240 with 147 mg of Dextrose, followed by filtering through a 0.45 μm filter.

### Preparation of Formulation 30-E (1% Compound (V)/5% PL90H/0.2% HPMC/3.5% Dextrose)

181.82 mg of Compound (VI) was dispersed using 6.7 g of a 0.5% HPMC (40-60 cps) in SWFI and 102 μL of a 5 N HCl, while mixing and heating (˜50° C.) until clear. Then 8.2 g of a 9% hydrogenated soy PC (PL90H) suspension in SWFI was added and sonicated, then 60 mL of a 2 N NaOH solution to adjust pH between 5.3-5.8. The composition was homogenized using sonicator probe (model GE-130), then osmolality adjusted to approximately 260 mOsm with 491 mg of dextrose (EP/BP/USP grade, Fisher Scientific), and filtered through a 0.22 μm PVDF syringe filter (Millipore).

### Preparation of Formulation 30-F (1% Compound (V)/0.2% Tyloxapol/0.3% HPMC/3.5% Dextrose)

186.15 mg of Compound (VI) was dispersed using 10.96 g of a 0.5% HPMC (40-60 cps) in SWFI and 102 μL of a 5 N HCl, while mixing and heating (˜50° C.) until clear. Then 3.123 g of a 1% Tyloxapol solution and 210 μL of a 2 N NaOH solution were added to adjust pH between 5.0-5.5. The composition was homogenized using sonicator probe (model GE-130), then osmolality adjusted to approximately 260 mOsm with 493.6 mg of dextrose (EP/BP/USP grade, Fisher Scientific), and filtered through a 0.22 μm PVDF syringe filter (Millipore). Formulations 30-A through 30-F prepared as described above were then tested and evaluated.

### EXAMPLE 31

### Ocular Tolerance of Formulated Compound (VI)

### Preparation of Formulation for Compound (V) (1% Compound (VI)/1% HPMC/3.5% Dextrose/0.2% Tyloxapol/0.005% BAK/0.025% EDTA)

989 mg of compound VI was dispersed using 55 g of a 0.5% HPMC (40-60 cps) in SWFI and 529 μL of a 5 N HCl, while mixing and heating (˜50° C.) until clear. Then 87.5 g of a 3.5 mg/mL Tyloxapol solution in 0.5% HPMC was added, osmolality was adjusted to approximately 256 mOsm with 5.18 g of dextrose (EP/BP/USP grade, Fisher Scientific), and 1081 μL of a 2 N NaOH solution was added to adjust pH between 5.0-5.5. The product was homogenized using the Avestin C5, then filtered through a 0.45 μm filter followed 0.22 μm PES syringe filter (Millipore). 516 μL of a 1% BAK solution and 516 μL of 5% EDTA were added to 103.05 g of formulation.

### Preparation of Vehicle (0.012% Carminic Acid in 1% HPMC/3.5% Dextrose/0.2% Tyloxapol/0.005% BAK/0.025% EDTA)

55.24 g of a 1% HPMC (40-60 cps) was mixed in SWFI and 529 μL of a 5 N HCl. Then 87 g of a 0.35% Tyloxapol solution in 1% HPMC and 1324 μL of a 2 N NaOH solution were added. 18.53 mg of carminic acid was added and pH was adjusted to 7.4 with 1N NaOH, then the osmolality was adjusted to 246 with 5.16 g of dextrose. 738 μL of 1% BAK and 738 μL of 5% EDTA were added to 147 g of solution to adjust pH to 7.4, followed by filtering through a 0.22 PES filter. A 0.9% saline solution was used as is (B/Braun) as a negative control.

### EXAMPLE 32

### Ocular Delivery of a Series of Compounds to the Back of the Eye of C57b1/6 Mice Via Topical Administration (Eve Drops)

A series of compounds were formulated as 1% drug substance in 0.2% Tyloxapol/1% HPMC made iso-osmotic with dextrose. The pH of the formulations ranged from 5-7.4 depending on the characteristics of each compound. The formulations were administered to c57b1/6 mice via topical administration and the amount of drug substance was analyzed at 2 and 7 hours after the last administration. The tissues were extracted and assayed by LC/MS/MS.

Although the invention has been described with reference to the above examples, it will be understood that modifications and variations are encompassed within the spirit and scope of the invention. Accordingly, the invention is limited only by the following claims.

What is claimed is:

1. A composition comprising:
a drug or its prodrug and a pharmaceutically acceptable carrier for ophthalmic delivery, wherein the drug has:
a) a polar surface area not exceeding about 150 Å²;
b) a water solubility of less than about 0.1 mg/mL at a pH range of 4-8;
c) a cLogD of at least about 0.5 at pH of 7.4; and
d) a molecular weight not exceeding about 1,000 Daltons,
with the proviso that the drug is not a steroidal molecule.

2. The composition of claim 1, wherein the drug has the polar surface area not exceeding about 120 Å².

3. The composition of claim 2, wherein the drug has the polar surface area not exceeding about 100 Å².

4. The composition of any one of claims 1-3, wherein the drug has a water solubility of less than about 0.05 mg/mL.

5. The composition of any claim 4, wherein the drug has a water solubility of less than about 0.01 mg/mL.

6. The composition of claim 1, wherein the drug has a cLogD of at least about 1.

7. The composition of claim 1, wherein the drug has a cLogD of at least about 2.

8. The composition of claim 1, wherein the drug has the molecular weight not exceeding about 900 Daltons.

9. The composition of claim 1, wherein the drug has the molecular weight not exceeding about 800 Daltons.

10. The composition of claim 1, wherein the drug or prodrug is selected from the group consisting of antiallergics, antirmigraine, antianemics, bronchodilators, analgesics, antibiotics, leukotriene inhibitors or antagonists, antihistamines, non-steroidal anti-inflammatories, antineoplastics, anticholinergics, anesthetics, anti-tuberculars, cardiovascular agents, lectins, peptides, and combinations thereof.

11. The composition of claim 1, wherein the drug or prodrug is a kinase inhibitor.

12. The composition of claim 11, wherein the kinase is selected from a group consisting of the Janus family kinases (Jak), the Src family kinases, the VEGF receptor family kinases, the PDGF receptor family kinases, the Eph receptor family kinase, and the FGF receptor family kinases.

13. The composition of claim 1, wherein the drug or prodrug is lipophilic.

14. The composition of claim 1, wherein the formulation is delivered to the back of the eye, intravitreally or periocularly.

15. The composition of claim 1, wherein the formulation is an eye drop formulation.

16. The composition of claim 1, further comprising a compound selected from the group consisting of an antiviral agent, an antibiotic, an intraocular pressure reducing composition, a wetting agent, a cataract prevention agent, a VEGF receptor inhibitor, an anti-inflammatory agent, an oxygen radical scavenger agent, and an NO inhibitor.

17. The composition of claim 1, further comprising a surface active component.

18. The composition of claim 17, wherein the surface active component is selected from a group consisting of phospholipids, phosphatidylcholines, phosphatidylethanolamines, cardiolipins, fatty acids, phosphatides, and non-ionic surfactants.

19. The composition of claim 18, wherein the non-ionic surfactants are selected from a group consisting of tyloxapol, polyethylenglycols and derivatives, like PEG400, PEG1500, PEG20000, poloxamer 407, poloxamer 188, tween 80, and polysorbate 20.

20. The composition of claim 1, wherein the drug or prodrug is not useful for treating glaucoma.

21. The composition of claim 18, wherein the drug or prodrug is not useful for treating glaucoma.

22. The composition of claim 1, wherein the composition is suitable for treatment pathological conditions of the eye selected from the group consisting of age-related macular degeneration, diabetic retinopathy, diabetic macular edema, cancer, and glaucoma.

23. The composition of claim 1, wherein the drug or its prodrug comprises a compound having structure A: 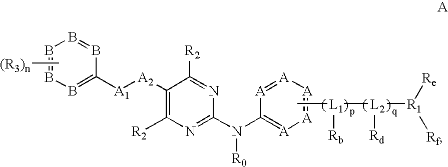
wherein each of A is independently selected from a group consisting of CH, N, NH, O, and S, or A is a part of a ring fusion to form a second ring, wherein the second ring is a ring selected from a group consisting of an aromatic, a heteroaromatic, a bicyclic aromatic, and a bicyclic aromatic heterocyclic ring;
each of B is, independently CH, or a part of a ring fusion to form a second ring, wherein the second ring is a ring selected from a group consisting of an aromatic, a bicyclic aromatic, and a bicyclic, with only the first ring being aromatic;
A₁ is selected from a group consisting of NRₐ, C(O), S(O), S(O)₂, P(O)₂, O, S, and CRₐ, wherein R is selected from a group consisting of H, a lower alkyl, a branched alkyl, a hydroxyalkyl, an aminoalkyl, a thioalkyl, an alkylhydroxyl, an alklythiol, and an alkylamino, and wherein if A₁ is NRₐ, then a=1, and if A₁ is CRₐ, then a=2;
A₂ is selected from a group consisting of NR, C(O), S(O), S(O)₂, P(O)₂, O, and S, with the proviso that the connectivity between A₁ and A₂ is chemically correct;
R₀ is selected from a group consisting of H, a lower alkyl, and a branched alkyl;
L₁ is selected from a group consisting of a bond, O, S, C(O), S(O), S(O)₂, NRₐ, and a C₁-C₆ alkyl; L₂ is selected from a group consisting of a bond, O, S, C(O), S(O), S(O)₂, a C₁-C₆ alkyl, and NRₐ; or L₁ and L₂ taken together form a bond;
each of R_{b}, R_{d}, Rₑ, and R_{f} either is absent or is independently selected from a group consisting of H, a C₁-C₆ alkyl, a cycloalkyl, a branched alkyl, a hydroxy alkyl, an aminoalkyl, a thioalkyl, an alkylhydroxyl, an alkylthiol, and an alkylamino;
each of p, q, m, r is independently an integer having value from 0 to 6;
R_{b} and R_{d} taken together form a moiety selected from a group consisting of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NRₐ—(CH₂)ₘ, and (CH₂)ᵣ—O—(CH₂)ₘ; or
R_{b} and Rₑ taken together form a moiety selected from a group consisting of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NRₐ—(CH₂)ₘ, and (CH₂)ᵣ—O—(CH₂)ₘ; or
R_{d} and R_{f} taken together form a moiety selected from a group consisting of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NRₐ—(CH₂)ₘ, and (CH₂)ᵣ—O—(CH₂)ₘ; or
R_{b} and R_{f} taken together form a moiety selected from a group consisting of (CH₂)ₘ, (CH₂)ᵣ—S—(CH₂)ₘ, (CH₂)ᵣ—SO₂—(CH₂)ₘ, (CH₂)ᵣ—SO₂—(CH₂)ₘ, (CH₂)ᵣ—NRₐ—(CH₂)ₘ, and (CH₂)ᵣ—O(CH₂)ₘ; or
R_{d} and Rₑ taken together form a moiety selected from a group consisting of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NRₐ—(CH₂)ₘ, and (CH₂)ᵣ—O—(CH₂)ₘ;
R₁ is selected from a group consisting of (CRₐ)ₘ, O, N, S, C(O)(O)R′, C(O)N(R′)₂, SO₃R′, OSO₂R′, SO₂R′, SOR′, PO₄R′, OPO₂R′, PO₃R′, PO₂R′, and a 3-6 membered heterocycle with one or more heterocyclic atoms, wherein R′ is selected from a group consisting of hydrogen, a lower alkyl, and an alkyl-hydroxyl, or R′ is a moiety selected from a group consisting of a closed 3-6 membered heterocycle with one or more heterocyclic atoms, a branched alkyl, and a branched alkyl hydroxyl, wherein each R′ is independent in case there is more than one R′;
R₂ is selected from a group consisting of hydrogen, an alkyl, a branched alkyl, phenyl, a substituted phenyl, halogen, an alkylamino, an alkyloxo, CF₃, sulfonamido, a substituted sulfonamido, an alkyoxy, a thioalkyl, a sulfonate, a sulfonate ester, phosphate, a phosphate ester, phosphonate, a phosphonate ester, carboxo, amido, ureido, a substituted carboxo, a substituted amido, a substituted ureido, and a 3-6 membered heterocycle with one or more hetrocyclic atoms, with the further proviso that either one or two substituents R₂ can be present in the ring, and if more than one substituent R₂ are present, each of the substituents is the same or different;
R₃ is selected from a group consisting of hydrogen, an alkyl, a branched alkyl, an alkoxy, a halogen, CF₃, cyano, a substituted alkyl, hydroxyl, an alklylhydroxyl, thiol, an alkylthiol, a thioalkyl, amino, and an aminoalkyl; and
n is an integer having value between 1 and 5, with the further proviso that if n≧2, then each group R₃ is independent of the other groups R₃,
and pharmaceutically acceptable salts, hydrates, solvates, crystal forms, N-oxides, and individuals diastereomners thereof

24. The composition of claim 23, wherein the drug or its prodrug is selected from a group consisting of compounds I, II, and III:

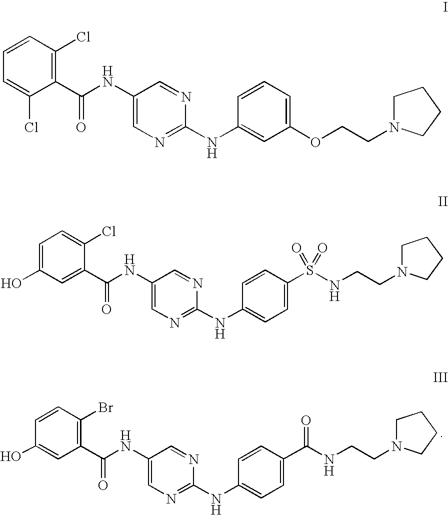

25. The composition of claim 24, wherein the drug or its prodrug is compound I:

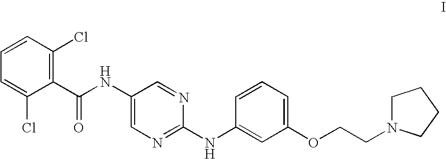

26. The composition of claim 24, wherein the drug or its prodrug is compound II:

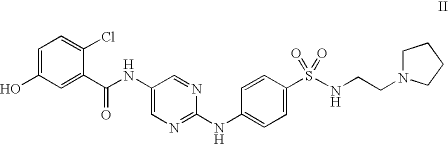

27. The composition of claim 24, wherein the drug or its prodrug is compound III:

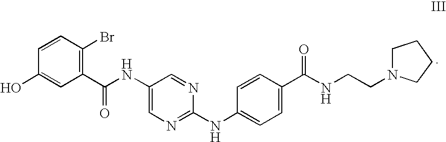

28. The composition of claim 1, wherein the drug or its prodrug comprises a compound having structure B: 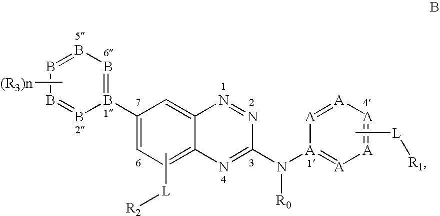
wherein:
wherein each of A is independently selected from a group consisting of (CH)₀₋₁, N, NH, O, S, and a part of a ring fusion to form a second ring, where the second ring is an aromatic, a heteroaromatic, a bicyclic aromatic, a bicyclic aromatic heterocyclic ring, or a bicyclic with only the first ring being aromatic or heteroaromatic;
each of B is independently selected from a group consisting of (CH)₀₋₁, N, NH, O, S, and a part of a ring fusion to form a second ring, where the second ring is an aromatic, a heteroaromatic, a bicyclic aromatic, a bicyclic aromatic heterocyclic ring, or a bicyclic with only the first ring being aromatic or heteroaromatic, with the further proviso that if each B is (CH)₀, R₃ is bonded directly to the adjacent ring.
R₀ is selected from a group consisting of H and a lower alkyl;
L is selected from a group consisting of a bond and a substituted or unsubstituted alkyl, alkenyl, or alkynyl linking moiety;
R₁ is selected from a group consisting of C(R′)₃, OR′, N(R′)₂, NR′C(O)R′, NR′C(O)O(R′), NR′C(O)N(R′)₂, SR′, C(O)(O)R′, C(O)R′, C(O)N(R′)₂, SO₃R′, OSO₂R′, SO₂R′, SOR′, S(O)N(R′)₂, OS(O)(O)N(R′)₂, S(O)(O)N(R′)₂, S(O)N(R′)₂, PO₄R′, OPO₂R′, PO₃R′, PO₂R′, and a 3-6 membered heterocycle with one or more heterocyclic atoms with each heteroatom independently being capable of carrying any R′ group on it, wherein R′ is selected from a group consisting of hydrogen, a lower alkyl, a substituted alkyl, an alkyl-hydroxyl, a substituted alkyl-hydroxyl, a thiol-alkyl, a thiol-substituted alkyl, an alkyl-thiol, a substituted alkyl-thiol, an aminoalkyl, an amino-substituted alkyl, an alkylamino, a substituted alkyl-amino, a branched alkyl, a branched substituted alkyl, a branched alkyl hydroxyl, a branched substituted alkyl hydroxyl, a branched thio-alkyl, a branched thio-substituted alkyl, a branched alkyl-thiol, a branched substituted alkyl-thiol, a branched aminoalkyl, a branched amino-substituted alkyl, a branched alkylamino, a branched substituted alkyl-amino, and a closed 3-6 membered carbocycle or heterocycle, wherein a substitutent in any of said substituted alkyls includes said closed 3-6 membered carbocycle or heterocycle, with the further proviso that each heteroatom in the 3-6 membered heterocycle is capable of carrying any R′ group on it, with the further proviso that the substitution in any of said substituted alkyls includes any R′ group connected to said alkyls via an atom other than carbon or via carbon, and wherein each R′ is independent in case there is more than one R′;
R₂ is a substitutent situated at position 5, 6 or 8 of the ring, wherein R₂ is selected from a group consisting of methyl, ethyl, n-propyl, iso-propyl, n-butyl, iso-butyl, tert-butyl, iso-pentyl, phenyl, substituted phenyl, halogen, a branched or unbranched alkylamino, a branched or unbranched aminoalkyl, a branched or unbranched alkyloxo, a branched or unbranched oxyalkyl, a branched or unbranched thioalkyl, a branched or unbranched alkylthiol, CF₃, sulfonamido, a substituted sulfonamido, sulfonate, a sulfonate ester, phosphate, a phosphate ester, a phosphonate, a phosphonate ester, carboxo, amido, ureido, a substituted carboxo, a substituted amido, a substituted ureido, or a 3-6 membered carbocycle or heterocycle attached to positions 5, 6 or 8 directly or through group L, each heteroatom independently being capable of carrying any group R₂, with the further proviso that either one, two or three substituents R₂ are present in the ring, each of the substituents R₂ being the same or different;
R₃ selected from a group consisting of hydrogen, an alkyl, an alkoxy, halogen, CF₃, cyano, a substituted alkyl, hydroxyl, an aryl, a substituted aryl, a heteroaryl, a substituted heteroaryl, a heterocycle, C(R″)₃, OR″, N(R″)₂, NR″C(O)R″, NR″C(O)NR″, R″, C(O)(O)R″, OC(O)R″, C(O)N(R″)₂, C(O), C(O)R″, OC(O)N(R″)₂, SO₃R″, OSO₂R″, SO₂R″, SOR″, PO₄R″, OPO₂R″, PO₃R″, PO₂R″, wherein R″ is hydrogen, an aryl, a substituted aryl, a heteroaryl, a substituted heteroaryl, a lower alkyl, a branched lower alkyl, an alkyl-hydroxyl, a branched alkyl-hydroxyl, an amino-alkyl, a branched amino-alkyl, an alkyl-amino, a branched alkyl-amino, a thiol-alkyl, a branched thiol-alkyl, an alkyl-thiol, a branched thiol-alkyl, or forms a closed 3-6 membered heterocycle with one or more heterocyclic atoms, a branched alkyl, a branched alkyl hydroxyl, wherein each R″ is independent in case there is more than one R″;
n is an integer having the value between 1 and 5, with the further proviso that if n≧2, then each group R₃ is independent of the other groups R₃,
with the further proviso that if each A is (CH)₀, L is a bond,
with the further proviso that if each B is (CH)₀, R₃ is any substitutent described above, other than hydrogen, bonded directly to the position 7 of the adjacent ring;
and pharmaceutically acceptable salts, hydrates, solvates, crystal forms, N-oxides, and individuals diastereomners thereof.

29. The composition of claim 28, wherein the drug or its prodrug comprises a compound selected from a group consisting of compounds IV-XX:

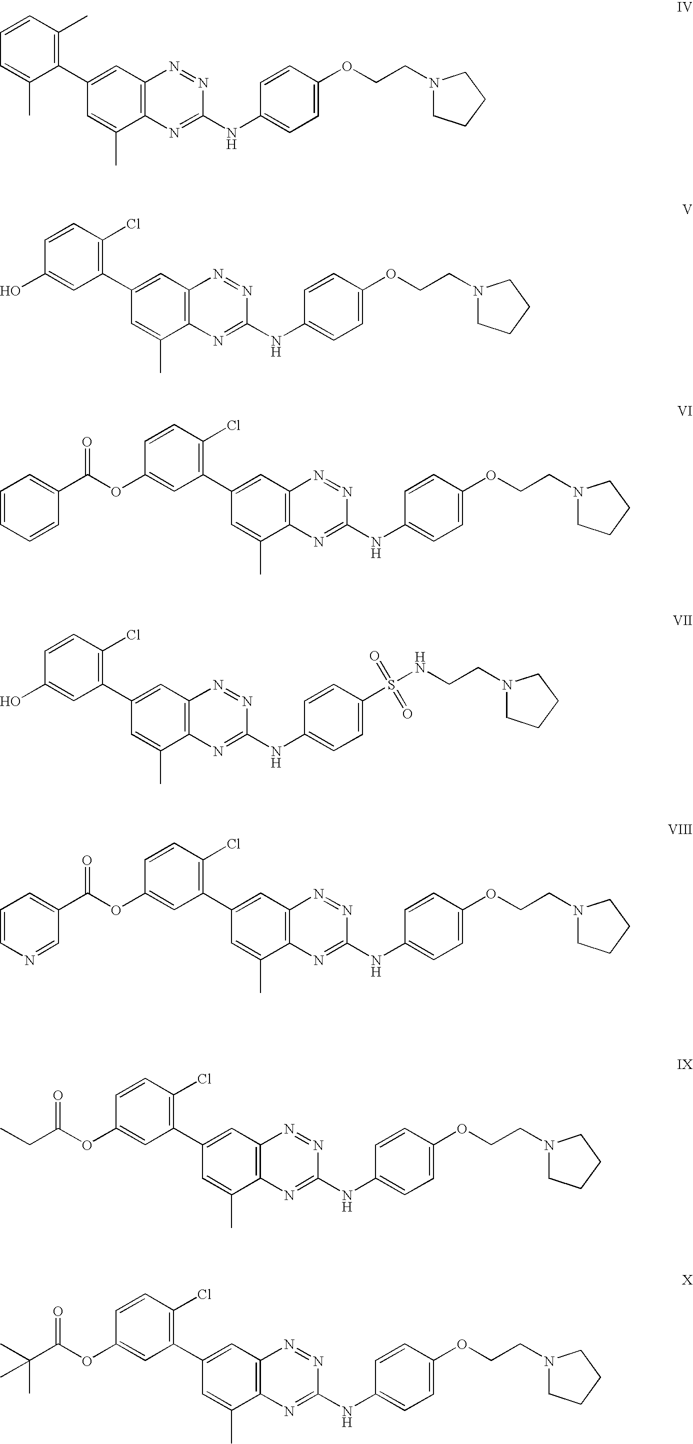

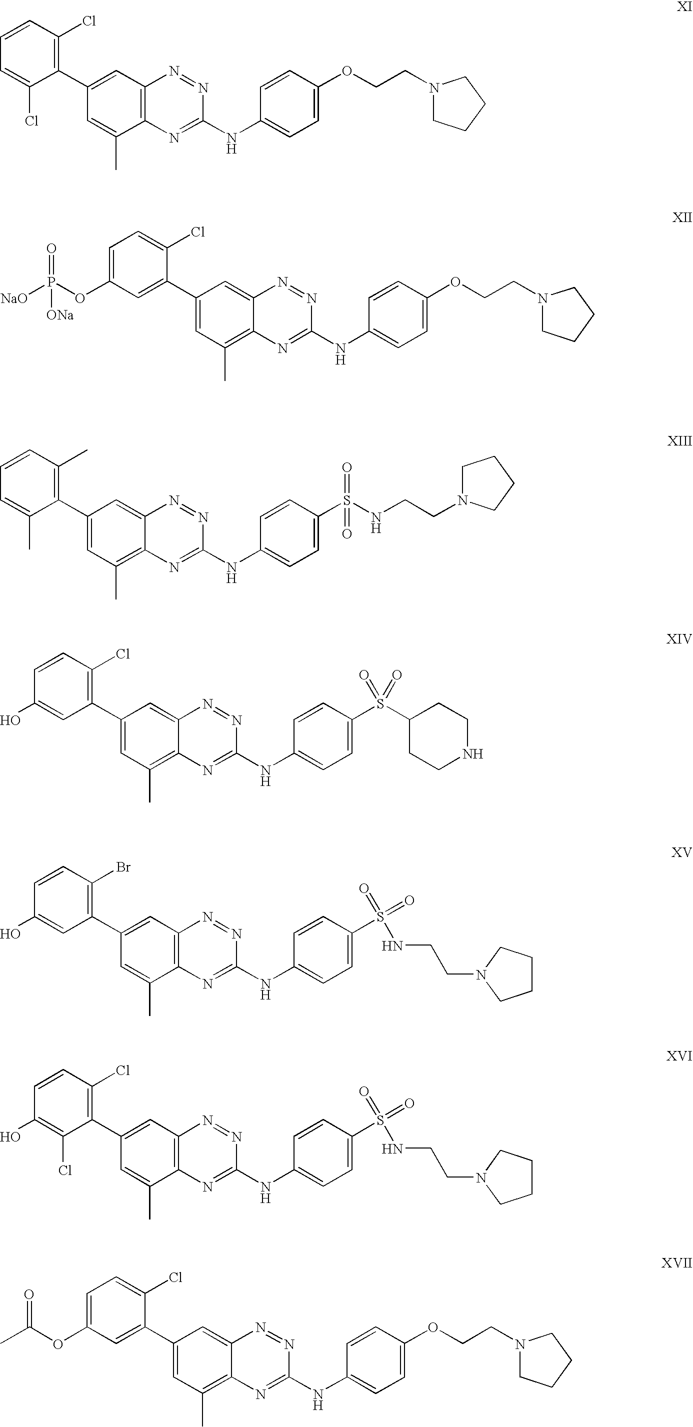

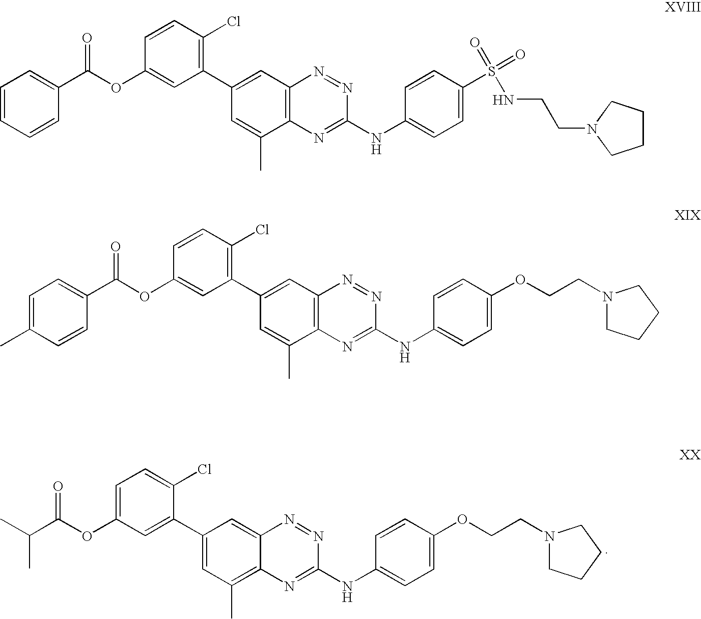

30. The composition of claim 28, wherein the drug or its prodrug comprises compound V:

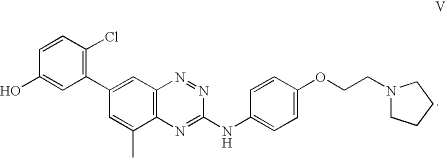

31. The composition of claim 28, wherein the drug or its prodrug comprises compound VI:

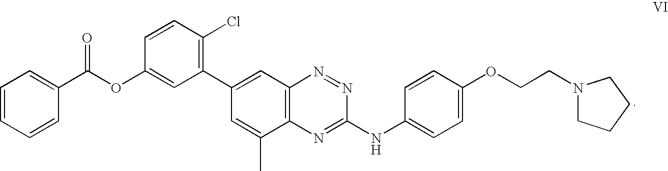

32. The composition of claim 28, wherein the drug or its prodrug comprises a compound selected from a group consisting of compounds VIII, IX, X, XVII, and XIX:

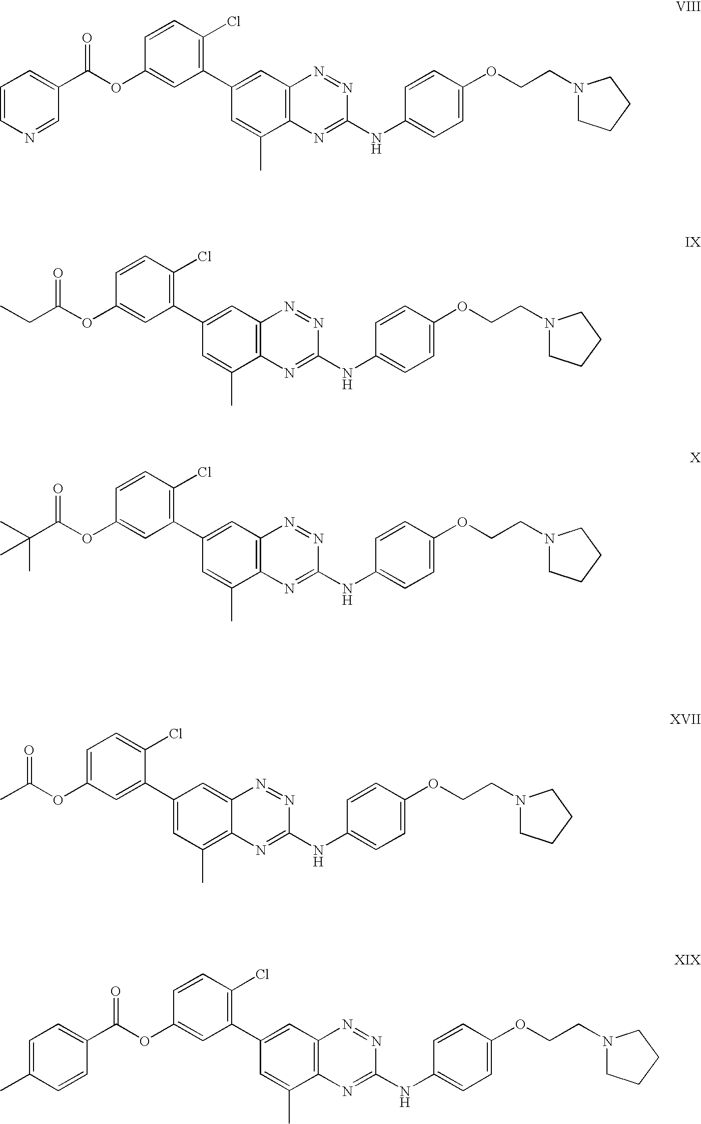

33. The composition of claim 28, wherein the drug or its prodrug comprises compound XII:

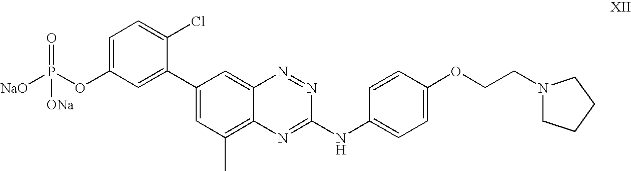

34. The composition of claim 28, wherein the drug or its prodrug comprises a compound selected from a group consisting of compounds VII, XV, and XVI:

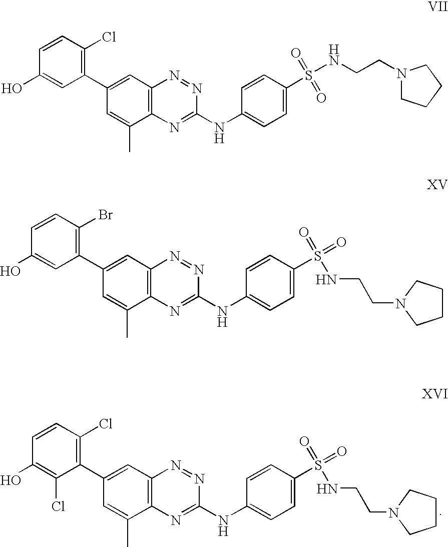

35. The composition of claim 28, wherein the drug or its prodrug comprises compound XIV:

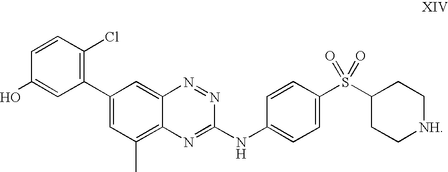

36. The composition of claim 28, wherein the drug or its prodrug comprises compound XVIII:

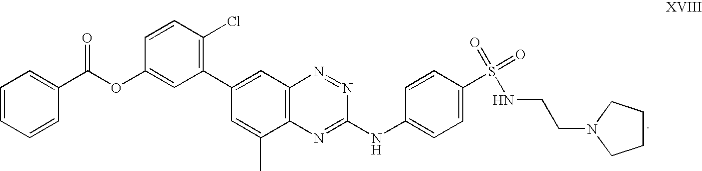

37. The composition of claim 28, wherein the drug or its prodrug comprises compound XI:

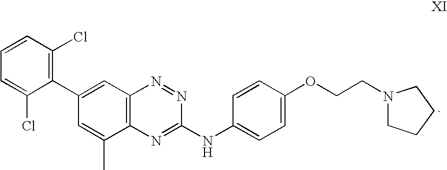

38. The composition of claim 1, further comprising a polymer based carrier capable of forming a colloidal suspension with the drug or prodrug.

39. The composition of claim 38, wherein the polymer is a lyophilic polymer.

40. The composition of claim 38, wherein the polymer is selected from the group consisting of cellulose derivatives, amylopectins and derivatives thereof, dextran and derivates thereof, poly(vinyl pyrrolidone), poly(vinyl alcohol), derivatives of poly(acrylic acid), derivatives of poly(methacrylic acid), and combinations thereof.

41. The composition of claim 40, wherein the cellulose derivatives are selected from a group consisting of methyl cellulose, hydroxyethyl cellulose, hydroxypropylmethyl cellulose, carboxymethyl cellulose, starches, amylase, and derivatives thereof.

42. A method for treating an ophthalmological condition in a subject comprising administering to a subject in need thereof a therapeutically effective amount of a composition of claim 1, thereby treating the condition.

43. The method of claim 42, wherein the ophthalmological condition is selected from the group consisting of age-related macular degeneration, diabetic retinopathy, diabetic macular edema, cancer, and glaucoma.

44. The method of claim 42, wherein the composition is administered to the back of the eye, intravitreally, or periocularly.

45. The method of claim 42, wherein the composition is in a formulation in the form of eye-drops.

46. The method of claim 45, wherein the composition is administered to the surface of the eye from about 1 to 4 times per day or per week.

47. The method of claim 42, wherein the composition is administered to a subject having dry age-related macular degeneration.

48. The method of claim 42, wherein the composition is administered to reduce the risk of progression of the ophthalmological disease.

49. The method of claim 42, further comprising administering a pharmaceutically acceptable substance selected from the group consisting of an antiviral agent, an antibiotic, an intraocular pressure reducing composition, a wetting agent, a cataract prevention agent, a VEGF receptor antagonist, an anti-inflammatory agent, an oxygen radical scavenger agent, and an NO inhibitor.

50. The method of claim 42, wherein the pharmaceutically acceptable substance is a VEGF receptor inhibitor.

51. The method of claim 42, further comprising administering a molecule selected from the group consisting of an RNAi molecule, an antisense molecule, a peptide, a small molecule compound, a polynucleotide and a protein.

52. The method of claim 42, wherein the composition is configured in a formulation selected from a group consisting of a solution, a gel, a suspension, an emulsion, and an ointment.

53. The method of claim 42, wherein the composition further comprises a pharmaceutically acceptable substance selected from a group consisting of a tonicity agent, a comfort-enhancing agent, a solubilizing aid, a antioxidant and a stabilizing agent.

54. A method for preparing a composition of claim 1 comprising:
dissolving or partially dissolving the drug in the presence or absence of an organic solvent;
mixing with an aqueous colloidal suspension containing the polymer base carrier with or without a surface active component;
optionally removing the organic solvent, if appropriate;
adding osmotic agents; and
adjusting pH to a value making the composition suitable for administration.

55. A method for preparing a composition of claim 1 comprising:
mixing the drug or prodrug with an aqueous colloidal suspension containing a polymer base carrier to form a colloidal suspension with a mean particle size less than 5 μm;
adding osmotic agents; and
adjusting pH to a value making the composition suitable for administration.

56. The method as in any one of claims 54 and 55, further comprising at least one of the following:
adding aseptic filling;
sterilization by filtering or autoclaving;
freeze-drying;
spray-drying; or
reconstitution of dry formulation before usage.

57. An article of manufacture comprising a vial containing a composition of claim 1.

58. The article of manufacture of claim 57, further comprising instructions for administration of the composition.

59. A method of delivering a compound to the back of an eye, comprising preparing a formulation comprising the composition of claim 1 and delivering the formulation to the eye of a subject in need of such delivery.

60. The method of claim 59, wherein the formulation is in the form of eye drops.

61. The method of claim 59, wherein the composition comprises a kinase inhibitor.

62. The method of claim 61, wherein the kinase is selected from a group consisting of a Src family kinase, a VEGF receptor family kinase, a PDGF receptor family kinase, an Eph receptor family kinase, an FGF receptor family kinase, and a Janus family kinase.

63. A method of identifying a compound suitable for delivery to the back of the eye, comprising:
(a) administering a compound by eye drop administration; and
(b) observing the distribution of the compound in the eye following eye drop administration, wherein the compound is a drug or prodrug of claim 1,
thereby identifying a compound suitable for delivery to the eye.
